(19) United States
(12) Patent Application Publication
(10) Pub. No.: US 2010/0040609 A1
(43) Pub. Date: Feb. 18, 2010

(54) METHODS FOR PREVENTING, POSTPONING OR IMPROVING THE OUTCOME OF INVASIVE SPINAL PROCEDURES

(76) Inventor: James R. Gorman, Wellesley, MA (US)

Correspondence Address: FISH & RICHARDSON P.C., PO BOX 1022, MINNEAPOLIS, MN, 55440-1022, US

(21) Appl. No.: 12/307,900

(22) PCT Filed: Jul. 9, 2007
(86) PCT No.: PCT/US07/73067

§ 371 Date: May 22, 2009

Related U.S. Application Data
(60) Provisional application No. 60/819,555, filed on Jul. 7, 2006.
(60) Provisional application No. 60/847,493, filed on Sep. 27, 2006.

(51) Int. Cl. A61K 39/395; A61K 38/17; A61K 31/7088

(52) U.S. Cl. 424/133.1; 424/130.1; 514/12; 514/44 R; 514/44 A

(57) ABSTRACT

Methods for identifying subjects who could benefit therapeutically from administration of a targeted anti-inflammatory therapy (TAT) are provided. Subjects that are identified include those that are eligible, based on pre-determined criteria, for a spinal surgery procedure, such as a laminectomy or diskectomy. Methods of preventing such procedures or improving the outcome of such procedures are also provided, and include administering a TAT to the subject by any route or regimen of administration, including both known and novel regimens described herein.

### CROSS-REFERENCE TO RELATED APPLICATIONS

This application claims priority under 35 U.S.C. §119 to U.S. Provisional Application Ser. Nos. 60/819,555, filed Jul. 7, 2006; and 60/847,493, filed Sep. 27, 2006, the contents of which are incorporated herein in their entireties.

This application is related to U.S. application Ser. Nos. ______ (Attorney Docket No. 21782-006001) and ______ (Attorney Docket No. 21782-007001), both filed concurrently herewith on Jul. 9, 2007, the entire contents of which are incorporated by reference herein in their entirety.

### TECHNICAL FIELD

This disclosure is related to identifying subjects who are currently treated preferentially by a spinal surgery procedure, but who, contrary to current teaching and practice, are surprisingly likely to benefit from treatment with a targeted anti-inflammatory therapy (TAT), such as an inflammatory cytokine inhibitor (IC-I), or an inflammatory mediator inhibitor (IM-I). The disclosure also relates to methods for preventing, reducing, postponing, delaying or eliminating the need for spinal surgery procedures such as diskectomy in patients with herniated disk (HD), or laminectomy in patients with spinal stenosis (SS). The disclosure also relates to methods for improving the therapeutic outcome of these invasive spinal procedures in the same patients. More particularly, this disclosure relates to the use of TATs, including TNF-α (TNF) inhibitors (TNF-Ts), administered either by known or novel regimens, in subjects who have met at least one predetermined standard of eligibility (SOE) for a spinal surgery procedure, such as diskectomy or laminectomy, that does not involve implantation of a device or intervertebral fusion. Subjects are identified as likely to benefit from TAT therapy by meeting at least one SOE for a spinal surgery procedure. Typically, the subject will meet the criteria of eligibility for the spinal surgery procedure in at least one relevant professional clinical practice guideline (CPG), which criteria will usually include confirmation of the HD or SS by appropriate imaging procedures such as MRI, the presence of moderate to severe persistent symptoms such as radiating pain for a defined period of weeks or months, and the failure to respond to conventional non-invasive therapies.

### BACKGROUND

Inflammatory Cytokines (ICs) and Inflammatory Mediators (IMs) in Diseases and Disorders

A wide variety of inducers can cause inflammation in the body, including trauma, injury, disease, surgery, infection and cytokines. Such stimuli can induce the production of IC by a wide variety of cells, including cells of the immune system, cells of the central and peripheral nervous systems and cells from other tissues and organs (FIG. 1). Certain IC, such as TNF, IL-1, IL-6, IL-8, IL-12, IL-15, IL-17, IL-18, IL-23, IFN-γ, GM-CSF, and MCP-1, play key roles in the induction and maintenance of inflammation. A subset of cytokines called chemokines, such as IL-8 and MCP-1, function in concert with other IC during inflammation to recruit cells from the blood or cerebrospinal fluid to the site of injury. A wide variety of cell types comprise the inflammatory cell infiltrate (FIG. 1). Cells recruited to the site of injury, particularly monocytes, macrophages and dendritic cells, produce additional IC which collectively modulate cell maturation, proliferation, activation and angiogenesis. These IC act on both infiltrating cells and local tissue cells to produce and release IM. Key IM include nitric oxide (NO), produced via activation of inducible NO synthase (iNOS), prostaglandinE2 (PGE2), an arachidonic acid metabolite resulting from the induction of the COX-2 enzyme, the matrix metalloproteinases (MMPs) MMP-1 (collagenase-1), MMP-2 (gelatinase A), MMP-3 (stromelysin), MMP-7 (matrilysin), MMP-9 (gelatinase B) and MMP-13 (collagenase-3), and the matrix-degrading aggrecanases ADAMTS4 and ADAMTS5 of the Adamalysin family of proteases. As illustrated in FIG. 1, IC and IM act individually and in concert to cause inflammation and tissue damage, for example in irritation, inflammation, and injury of the spinal nerve root (NR). They also cause degradation of proteoglycans and extracellular matrix, as in matrix destruction in intervertebral disks and cartilage.

### Role of ICs and IMs in Peripheral Neuropathic Injury and NR Injury

Spinal disorders such as HD and SS cause mechanical compression of spinal nerve roots (NRs) and nerves, initiating a biochemical cascade in which ICs such as TNF play an essential role. The resulting NR injury, when significant, causes radiating pain along the distribution of the affected NR. This “radicular pain” is colloquially known as “sciatica” when occurring in the lower back and radiating into the buttock, thigh, or leg, in the distribution of the sciatic nerve.

TNF and other ICs and IMs are increasingly implicated in controlling the pathophysiology of NR injury and resulting radiating pain. For example, TNF expression increases rapidly after nerve injury and stimulates expression of other ICs and IMs, including interleukin IL-1, IL-6, and IL-8, leading to increased neuronal excitability and neuro-inflammation.

Data from multiple preclinical models suggest that TNF inhibition can neutralize the pathophysiology of nerve injury and pain resulting from disk injury and can prevent or neutralize peripheral neuropathic injury and pain. The potential efficacy of IV or SC administered TNF-Is has also been tested in several preliminary human clinical trials in patients with HD and radiating pain, including one open label trial [1] and one blinded trial [2].

### Current Care of Spinal Disorders Such as HD and SS

Severe or persistent radicular pain is frequently associated with HD. In patients with HD in the lower back, persistent pain can originate in the back and often extends (“radiates”) into the leg along the distribution of the sciatic nerve (lumbar radicular pain, or sciatica). In patients with HD in the neck, the persistent pain can originate in the neck and often radiates into the arm. Patients can be diagnosed with HD through a variety of characteristic findings. These include, for example: a) persistent radiating pain; b) characteristic findings on a physical exam indicative of NR irritation, injury or inflammation, such as limited mobility or range of motion due to pain; c) abnormalities in the strength and sensation of particular parts of the body that are found with a neurological examination; d) radiologic confirmation of an HD at the appropriate level to explain symptoms of radiating pain, weakness, or numbness in the legs, back, arms, or neck, found upon MRI, CT, and CT myelography; e) electrodiagnostic studies that may differentiate peripheral neuropathies, determine the spinal NR level of the HD, and corroborate physical examination findings; and f) invasive procedures using a needle or other invasive technique, including diskography and provocative diskography, or partial removal of the nucleus pulposus or annulus fibrosus [3].

SS, either acquired or congenital, results from degenerative changes in the spine, variably including the intervertebral disks, the intervertebral joints (facet joints) and the ligamentum flavum. In each case, the degenerative changes together result in a gradual narrowing of the lumbar or cervical spinal canal, causing compression of the spinal cord and NRs. Symptoms include: a) pain and/or numbness in the neck, back, buttocks, legs, thighs or calves that is worse with walking, standing and/or exercise; b) back pain that radiates to the legs; c) weakness of the legs; and d) difficulty or imbalance when walking. Patients can be diagnosed with SS through, for example, a) persistent radiating pain; b) neurologic examination findings of abnormal sensation and muscle weakness in the legs; c) gait disturbances and characteristic bent over posture; d) asymmetric deep tendon reflexes; and e) radiologic findings of SS by x-ray (e.g., myelogram), MRI, spinal CT or CT myelography. Depending on whether the stenosis is central or foraminal, provocative maneuvers on physical examination such as side bending reproducing the pain may be negative or positive, respectively.

Patients diagnosed with HD or SS may receive an initial trial of conservative therapy including rest and behavioral modification, and oral analgesics to provide conventional anti-inflammatory therapy, such as non-steroidal anti-inflammatory drugs (NSAIDs) and oral glucocorticoids. When relief provided by conservative therapy proves inadequate, treatment typically progresses to opioid analgesics and to more invasive, expensive epidural injections of steroids or of local anesthetics (LAs), also called “nerve root blocks.” Even these invasive measures performed by sub-specialists including anesthesiologists, radiologists and spine surgeons, are often inadequate in the degree and/or durability of pain relief provided. For patients with confirmed HD and persistent radicular pain of 4-8 weeks duration, or stenosis and persistent radicular pain of 8-16 weeks duration, evaluation as to whether to proceed with diskectomy [3] or laminectomy [4] is recommended.

In current practice, many patients with HD or SS elect to undergo a spinal surgery procedure such as diskectomy or laminectomy. Patients meeting eligibility criteria for such invasive procedures are routinely offered surgical treatment as the standard of care, rather than drug therapies. Spinal disorders such as compression of the NR by an HD or SS are viewed as resulting from compressive or biomechanical forces, rather than by a biochemical imbalance potentially treatable with a targeted drug therapy. Patients with extensive HD or SS with associated severe or persistent pain are typically considered to be injured beyond the therapeutic abilities of non-invasive drug therapies, and thus to require surgical intervention to relieve the biomechanical imbalance in the spine. In contrast, patients considered as candidates for a drug therapy are typically those patients whose conditions are sufficiently non-severe to warrant recommendation for watchful waiting and non-invasive “conventional medical care,” rather than eligibility for an invasive spinal procedure.

The current standard of care does not teach administration of a TAT, such as a TNF-I, to patients diagnosed as eligible for an invasive spinal procedure. Such patients may be offered epidural steroids, but typically, epidural steroids will be part of the treatment that has failed in order to qualify the patient for surgery. In practice, once steroid treatments fail, the patients are considered eligible for surgery. It is typically thought that patients eligible for surgery will benefit from surgery rather than from administration of a TAT, such as the currently marketed TNF-Is Enbrel® (etanercept), Humira® (adalimumab), and Remicade® (infliximab).

Thus, TAT therapy including TNF-I therapy is not currently practiced in patients identified as eligible for spinal surgery procedures, or in patients who actually undergo a spinal surgery procedure. The efficacy and suitability of these agents for this class of patients is surprising. Indeed, as described below, current practice and teaching poses specific barriers to use of TNF-Is in patients found eligible for spinal surgery, and additional barriers in patients who actually undergo spinal surgery.

First, the currently marketed TNF-I compounds, Enbrel® (etanercept), Humira® (adalimumab), and Remicade® (infliximab), are protein therapeutics, either monoclonal antibodies or soluble cytokine receptor fusion proteins. Enbrel®, Humira®, and Remicade® are approved for use by systemic routes of administration, either IV or SC. Such agents are widely viewed as not crossing the blood brain barrier, and therefore likely of limited use in treating disorders of the spinal NR such as HD or SS. The disk itself is poorly vascularized and would not be expected to be substantially accessible to protein therapeutics administered by parenteral routes. There is little or no experience to guide the use of emerging TATs such as protein therapeutics by localized routes of administration such as epidural or intradiskal administration.

Second, treatment with the marketed TNF-Is has been linked with an increased risk of certain infections, a risk of significant potential concern to interventionalists such as spine surgeons. This perceived potential for increased risk of infection presents a barrier to TNF-I use in patients eligible for or scheduled for spine surgery. Chronic therapy with currently marketed TNF-Is is known to increase the risk of certain infections, particularly tuberculosis (TB). Other rarer, sometimes serious infections have also been associated with use of TNF-Is. Therefore, use of TNF-Is is contra-indicated in patients at high risk of infection, including patients with prior exposure to TB, with compromised immune systems, or with heightened risk of serious infection. Patients scheduled for or undergoing major surgery requiring exposure of deep musculoskeletal structures such as inter-vertebral disks are typically considered to be at heightened risk of serious infection. Many clinicians believe that TNF-I therapy may increase the risk of post-operative infection in surgery patients. While there is no convincing data to suggest that TNF-Is cause an increased risk of infection by the types of organisms found in post-operative infections in surgical patients, nevertheless, TNF-Is are typically not prescribed due to the concern regarding potential increased risk of infection. For example, for rheumatoid arthritis patients on chronic TNF-I therapy and undergoing joint replacement surgery, TNF-I therapy is often discontinued prior to surgery. Thus, current perceptions of TNF-Is and practice in management of perceived infection risk mitigate against use of TNF-Is and other emerging TATs in patients found eligible for a spinal surgery procedure.

Similarly, once a determination is made that the patient will actually undergo the procedure, TATs are not prescribed. The spinal surgery procedure is viewed as likely to alleviate the mechanical disorder. The inventor has discovered that even when a disk or lamina is removed, the procedure itself can further exacerbate the disorder, likely through activation of pathways that release ICs and IMs. Thus, patients undergoing a spinal surgery procedure are surprisingly, likely to benefit from an administration of a TAT such as a TNF-I, through improved outcome of the spinal surgery procedure.

In summary, many patients with a spinal disorder such as HD or SS who fail to respond to conventional non-invasive treatments will be found eligible for and will undergo a spinal surgery procedure such as diskectomy or laminectomy. These invasive procedures are limited by inherent risks, high failure rates, and uncertain outcome. For patients eligible for a spinal surgery procedure, a need exists for improved non-surgical methods to provide rapid and substantial pain relief, in order to prevent or delay, if possible, the need for the spinal surgery procedure. In addition, for patients who do undergo a spinal surgery procedure, there is a need for effective, safe treatments to reduce the damage caused by the surgery procedure itself.

The invention provides a conceptually simple but surprising method of identifying patients as candidates for therapy with a TNF-I or other TAT. Contrary to current literature, teaching and practice, the disclosure provides that eligibility for a spinal surgery procedure, rather than identifying a subject as inappropriate for therapy with a TNF-I or other TAT, specifically identifies a patient as likely to benefit from TAT including TNF-I therapy. Surprisingly, through practice of the invention, many patients eligible to undergo surgery will be able to avoid the need for surgery through practice of the invention. Moreover, for patients who do undergo a spinal surgery procedure, TAT therapy can improve the outcome and speed post-operative recovery.

### SUMMARY

The present disclosure is directed to identifying and treating subjects with spinal disorders such as HD or SS who currently are typically not offered treatment with a TAT such as a TNF-I, but who could in many or most cases benefit from TAT treatment. The inventor has made the surprising discovery that patients suffering from moderate to severe disorders of the spine who have been determined to be eligible for a spinal surgery procedure, such as a diskectomy or laminectomy (“decompression surgery”), are candidates for TAT treatment to prevent, delay, or improve the outcome of the spinal surgery procedure.

The inventor has discovered that a subject or class of subjects can be reliably identified as highly likely to benefit therapeutically from TAT treatment if the subject or subjects meet(s) one or more of the following SOE for a spinal surgery procedure:

a) a determination of eligibility of the subject for the spinal surgery procedure by a healthcare service provider, as evidenced by;
i) a scheduling or request for scheduling by a healthcare service provider of the spinal surgery procedure for the subject;
ii) a communication by a healthcare service provider to the subject that the subject has been determined to be eligible for the spinal surgery procedure by the healthcare service provider;
iii) a provision or offering by a healthcare service provider to the subject of a consent form for the spinal surgery procedure;
iv) a receipt or execution by the subject of a consent form offered by a healthcare service provider for the spinal surgery procedure;
v) a notation by the healthcare service provider in a tangible medium such as the patient's written or electronic medical record that the patient is eligible for the spinal surgery procedure;

b) a determination of eligibility of the subject for the spinal surgery procedure by a qualified entity other than the subject's healthcare provider;

c) the meeting by the subject of the eligibility criteria for a spinal surgery procedure in one or more generally accepted CPG(s);

d) eligibility of the subject for a diskectomy, as indicated by the subject meeting all of the following 3 clinical criteria:
i) the subject exhibits symptoms of radiating back, neck, arm, and/or leg pain for a period of at least 4 to 8 weeks;
ii) radiological (e.g., MR, CT, CT myelogram) determination of HD in the subject at the appropriate spinal location has been recorded; and iii) the subject exhibiting one or more of the following:
   aa) evidence of spinal NR irritation or spinal cord deterioration (myelopathy) based on physical examination and/or electrodiagnostic studies;
   bb) failure to respond adequately to one or more conventional non-invasive treatments; and
   cc) limitation in the ability to perform normal activities such as walking, standing, or finding pain free positions; and
e) eligibility of the subject for a laminectomy as indicated by the subject meeting all of the following 3 clinical criteria:
   i) the subject exhibits symptoms of radiating back, neck, arm, and/or leg pain for a period of at least 8 weeks to 16 weeks;
   ii) a radiological (e.g., MR, CT, CT myelogram) determination of HD or SS in the subject at the appropriate spinal location has been recorded; and
   iii) the subject exhibits one or more of the following:
      aa) evidence of spinal NR irritation or spinal cord deterioration (myelopathy) based on physical examination and/or electrodiagnostic studies;
      bb) failure to respond adequately to one or more conventional non-invasive treatments; and
      cc) limitation in the ability to perform normal activities such as walking, standing, or finding pain free positions.

The methods provided herein may thus be useful in preventing or postponing the need for a spinal surgery procedure, or in improving the outcome of the procedure. While not being bound by theory, these benefits could be caused by preventing or reducing moderate to severe symptoms of the spinal disorder, such as by inhibiting or blocking the effects of ICs or IMs. The disclosure may be further useful in preventing or reducing injury to or irritation of the spinal NR, dorsal root ganglion, or peripheral nerve. Thus, the methods described herein may be useful in inducing remission from the troubling symptoms, such as persistent radicular pain, which accompany the underlying pathologies of HD, SS, or related spinal disorders.

Accordingly, it is one object of the present invention to provide methods and materials for preventing, reducing, delaying, postponing, or eliminating the need for a spinal surgery procedure such as diskectomy or laminectomy, or for improving the outcome of such procedures, by treating or reducing the symptoms and disability necessitating surgery, such as NR irritation, inflammation, injury and resulting pain. In one embodiment, the method of the present invention comprises identifying subjects likely to benefit therapeutically from treatment with a TAT, e.g., a TNF-I, who heretofore would not have been treated with the same. Such subjects have met at least one established criterion to be eligible candidates for a spinal surgery procedure such as diskectomy or laminectomy. For example, the present methods can include identifying, as subjects likely to benefit from the therapies described herein (e.g., administration of a TNF-I), subjects with HD who are candidates for spinal surgery procedures according to the eligibility criteria in standard CPGs. Two widely referenced illustrative CPGs are the CPGs on management of HD and on management of SS, developed by the North American Spine Society (NASS) and the American Academy of Orthopedic Surgeons (AAOS), and published by NASS, and often referred to interchangeably as the “NASS Guidelines”, the “AAOS guidelines”, or the “NASS-AAOS guidelines” [3,4]. The NASS CPG on HD [3] recommends that patients meet the following criteria before undergoing diskectomy surgery: 1) persistent symptoms of radiating back and leg pain for 4 to 8 weeks; 2) MRI or CT or CT myelographic findings of HD at the symptomatic level and on the symptomatic side to explain the symptoms; 3) positive sign(s) of NR irritation on physical exam, such as reduced ability to raise the legs in a straight leg raise test; and 4) failure to respond adequately to conventional non-invasive treatments including bed rest, physical therapy, NSAIDs, and possibly opioid medications.

Therapy according to the invention consists of administration of a TAT, such as an IC-I or IM-I as described herein. The TAT is administered either by a standard regimen and/or route, or by a novel regimen, for example, a novel regimen as described herein. For example, the TAT could be administered using an intradiskal/peridiskal regimen, as described herein. In some cases, the TAT could be administered systemically, e.g., via IV, intramuscular, or SC injection. In other cases, a regimen could include administering (a) an induction regimen comprising a TAT (e.g., a TNF-I); and (b) a maintenance regimen comprising a TAT (e.g., a TNF-I). Any regimen can also involve temporary peri-operative interruption of the treatment course with the TAT, e.g., TNF-I, in order to reduce the perceived risk of post-operative infection, with resumption of the TAT treatment regimen post-operatively. Provided herein also are teachings of how to establish the proper timing and duration for peri-operative interruption of therapy at the discretion of the clinician responsible for managing the patient's therapy before, during, and/or after the spinal surgery procedure.

Described herein is a method of identifying a subject who could benefit therapeutically from administration of a direct TNF inhibitor (direct TNF-I), the method comprising determining that the subject meets at least one predetermined standard of eligibility (SOE) for a spinal surgery procedure, thereby identifying the subject as one who could benefit.

Also described herein is a method of identifying a subject who could benefit therapeutically from administration of an NFκB Inhibitor (NFκB-I), the method comprising determining that the subject meets at least one predetermined SOE for a spinal surgery procedure, thereby identifying the subject as one who could benefit.

The above methods include a subject diagnosed with HD and eligible for diskectomy, or a subject diagnosed with SS and eligible for laminectomy. The above described predetermined SOE is selected from a determination of eligibility of the subject for the spinal surgery procedure by a healthcare service provider, as evidenced by; a scheduling or request for scheduling by a healthcare service provider of the spinal surgery procedure for the subject; a communication by a healthcare service provider to the subject that the subject has been determined to be eligible for the spinal surgery procedure; a provision or offering by a healthcare service provider to the subject of a consent form for the spinal surgery procedure; a receipt or execution by the subject of a consent form for the spinal surgery procedure, said consent form provided by the subject's healthcare provider; or a notation by the healthcare service provider in a tangible medium that the patient is eligible for the spinal surgery procedure. The method further includes a determination of eligibility of the subject for the spinal surgery procedure by a qualified entity other than the subject's healthcare provider, the meeting by the subject of the eligibility criteria for a spinal surgery procedure in one or more CPG(s), eligibility of the subject for a diskectomy, (as indicated by the subject meeting all of the following 3 clinical criteria, the subject exhibits symptoms of radiating back, neck, arm, and/or leg pain for a period of at least 4 to 8 weeks; radiological (e.g., MR, CT, CT myelogram) determination of HD in the subject at the appropriate spinal location has been recorded; and the subject exhibiting one or more of the following: evidence of spinal nerve root (NR) irritation or spinal cord deterioration (myelopathy) based on physical examination and/or electrodiagnostic studies; failure to respond adequately to one or more conventional non-invasive treatments; and limitation in the ability to perform normal activities such as walking, standing, or finding pain free positions); and eligibility of the subject for a laminectomy as indicated by the subject meeting all of the following 3 clinical criteria: i) the subject exhibits symptoms of radiating back, neck, arm, and/or leg pain for a period of at least 8 weeks to 16 weeks; ii) a radiological (e.g., MR, CT, CT myelogram) determination of HD or SS in the subject at the appropriate spinal location has been recorded; and iii) the subject exhibits one or more of the following: evidence of spinal NR irritation or spinal cord deterioration (myelopathy) based on physical examination and/or electrodiagnostic studies; failure to respond adequately to one or more conventional non-invasive treatments; and limitation in the ability to perform normal activities such as walking, standing, or finding pain free positions.

The above methods further comprise recording the identification of the subject in a tangible medium, administering a direct TNF-I to the subject, or administering an NFκB-I to the subject. In an embodiment, the direct TNF-I is an antibody or antibody fragment, a fusion protein, a peptide, an SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, or a dominant negative TNF molecule. Alternatively, the direct TNF-I is Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); or a therapeutic human polyclonal anti-TNF or a anti-TNF-R antibodies (THP). In an embodiment, the aforementioned NFκB-I is sulfasalazine, sulindac, clonidine, helenalin, wedelolactone, pyrollidinedithiocarbamate (PDTC), IKK-2 inhibitors, or an IKK inhibitor.

In an embodiment, a method is described for preventing or postponing a spinal surgery procedure in a subject where the subject meets at least one predetermined SOE for a spinal surgery procedure. This method includes, a) optionally identifying the subject as a subject eligible for the spinal surgery procedure, b) administering to the subject a therapeutically effective amount of at least one direct TNF-I, and c) optionally determining whether the subject's eligibility for the spinal surgery procedure has been prevented or postponed. In an embodiment, the disclosure describes a method for preventing or postponing a spinal surgery procedure in a subject where the subject meets at least one predetermined SOE for a spinal surgery procedure. The method includes, a) optionally identifying the subject as a subject eligible for the spinal surgery procedure, b) administering to the subject a therapeutically effective amount of at least one NFκB-I, and c) optionally determining whether the subject's eligibility for the spinal surgery procedure has been prevented or postponed. Both of these methods may include a subject diagnosed with HD and that is eligible for diskectomy, or a subject diagnosed with SS and that is eligible for laminectomy. In an aspect, these methods include where the predetermined SOE is selected from: a) a determination of eligibility of the subject for the spinal surgery procedure by a healthcare service provider (as evidenced by: i) a scheduling or request for scheduling by a healthcare service provider of the spinal surgery procedure for the subject; ii) a communication by a healthcare service provider to the subject that the subject has been determined to be eligible for the spinal surgery procedure; iii) a provision or offering by a healthcare service provider to the subject of a consent form for the spinal surgery procedure; iv) a receipt or execution by the subject of a consent form for the spinal surgery procedure, said consent form provided by the subject's healthcare provider; or v) a notation by the healthcare service provider in a tangible medium that the patient is eligible for the spinal surgery procedure); b) a determination of eligibility of the subject for the spinal surgery procedure by a qualified entity other than the subject's healthcare provider; c) the meeting by the subject of the eligibility criteria for a spinal surgery procedure in one or more CPG(s); d) eligibility of the subject for a diskectomy (as indicated by the subject meeting all of the following 3 clinical criteria: i) the subject exhibits symptoms of radiating back, neck, arm, and/or leg pain for a period of at least 4 to 8 weeks; ii) radiological (e.g., MR, CT, CT myelogram) determination of HD in the subject at the appropriate spinal location has been recorded; and iii) the subject exhibiting one or more of the following: evidence of spinal NR irritation or spinal cord deterioration (myelopathy) based on physical examination and/or electrodiagnostic studies; failure to respond adequately to one or more conventional non-invasive treatments; and limitation in the ability to perform normal activities such as walking, standing, or finding pain free positions); and e) eligibility of the subject for a laminectomy (as indicated by the subject meeting all of the following 3 clinical criteria: i) the subject exhibits symptoms of radiating back, neck, arm, and/or leg pain for a period of at least 8 weeks to 16 weeks; ii) a radiological (e.g., MR, CT, CT myelogram) determination of HD or SS in the subject at the appropriate spinal location has been recorded; and iii) the subject exhibits one or more of the following: evidence of spinal NR irritation or spinal cord deterioration (myelopathy) based on physical examination and/or electrodiagnostic studies; failure to respond adequately to one or more conventional non-invasive treatments; and limitation in the ability to perform normal activities such as walking, standing, or finding pain free positions). In an aspect, these methods further comprise objectively or subjectively assessing the effect of administering to the subject a therapeutically effective amount of at least one direct TNF-I or an NFκB-I on the subject, where the assessment comprises at least one of the following steps: a) determining a level or temporal duration of pain, impaired mobility, disability, or spinal NR irritation in the subject; b) determining an amount of TNF in the subject at a location of interest; c) fluoroscopically or radiologically observing the subject; d) determining whether the subject continues to meet the eligibility criteria in the predetermined SOE or CPG for the spinal surgery procedure; e) determining a measure of disability using the Oswetry Disability Index; f) determining a measure of functioning using the Short Form 36 Assay; e) optionally comparing the results of any one of steps a) to f) with the results of the same step performed prior to the administering to the subject a therapeutically effective amount of at least one direct TNF-I or an NFκB-I. In an aspect, the method comprises administering at least 2 separate administrations of a direct TNF-I. In an alternative aspect, the method comprises administering at least 2 separate administrations of an NFκB-I. In an aspect, the direct TNF-I is administered locally to an HD or site of SS. In an aspect, the NFκB-I is administered locally to an HD or site of SS. The route of administration for the direct TNF-I or an NFκB is selected from the group consisting of intra-operative, intrathecal, intradiskal, peridiskal, epidural (including periradicular and transforaminal), any combination of intradiskal, epidural, and peridural, perispinal, IV, intramuscular, SC, oral, intranasal, inhalation, and transdermal. In an aspect, administering to the subject a therapeutically effective amount of at least one direct TNF-I treats the subject so that the subject does not undergo a spinal surgery procedure in at least the first three months after the initial administration of the TNF-I. In an aspect, administering to the subject a therapeutically effective amount of at least one NFκB-I treats the subject so that the subject does not undergo a spinal surgery procedure in at least the first three months after the initial administration of the NFκB-I.

In an embodiment, these methods further comprising performing the spinal surgery procedure on the subject. In an aspect, the method further comprises administering a direct TNF-I in a time period that is prior to, during, and/or after the time period of the spinal surgery procedure. In an aspect, the method further comprises administering an NFκB-I in a time period that is prior to, during, and/or after the time period of the spinal surgery procedure. In an aspect, the method further comprises administering a direct TNF-I according to a protocol that may be optionally interrupted for a time period prior to and/or after the spinal surgery procedure. In an alternative aspect, the method further comprises administering an NFκB-I according to a protocol that may be optionally interrupted for a time period prior to and/or after the spinal surgery procedure. In one aspect, the therapeutic outcome of the subject from the spinal surgery procedure is improved. In an alternative aspect, the improvement in therapeutic outcome includes at least one of the following: a) a reduction in one or more of the symptoms that rendered the subject eligible for the invasive procedure (e.g., the intensity or chronicity of the subject's radiating pain or radicular pain; the degree of the subject's impaired ability to perform activities of daily living; and the degree of the subject's neurologic impairment, muscle weakness, NR irritation); b) a reduction in the amount of a cytokine in the subject in a location of interest; c) an improvement in the abnormal findings previously observed on fluoroscopic or radiologic examination of the subject; d) the subject's no longer meeting the eligibility criteria in the predetermined SOE or CPG for the spinal surgery procedure; e) accelerated recovery of the subject from the spinal surgery procedure as evidenced by fewer days spent in the hospital in the post-operative period; f) an accelerated return of the subject to the activities of daily living; g) an increased quality of life of the subject; h) a decrease in the time to return to work for the subject; i) a decrease in the time to restoration of functional capabilities for the subject; and j) a reduced incidence of failed procedure, as evidenced by a reduced incidence of eligibility for a repeat or revision spinal surgery procedure.

In an embodiment, the direct TNF-I is selected from the group consisting of an antibody or antibody fragment, a fusion protein, a peptide, a SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, and a dominant negative TNF molecule. In another aspect, the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

In an embodiment, the NFκB-I is selected from the group consisting of sulfasalazine, sulindac, clonidine, helenalin, wedelolactone, pyrollidinedithiocarbamate (PDTC), IKK-2 inhibitors, and IKK inhibitors.

In a particular embodiment, the direct TNF-I is administered using an administration regimen that comprises: (a) an induction regimen comprising a direct TNF-I; and (b) a maintenance regimen comprising a direct TNF-I.

In a further particular embodiment the NFκB-I is administered using an administration regimen that comprises: (a) an induction regimen comprising an NFκB-I; and (b) a maintenance regimen comprising an NFκB-I. The method of claim 35 or 36, wherein the induction regimen is administered intrathecally, intradiskally, peridiskally, or epidurally, or combinations thereof.

For both of the above disclosed methods, the maintenance regimen comprises systemic or parenteral administration, IV, perispinal, intramuscular, SC, or transdermal administration, administration by a pump, and administration by implantation of a depot formulation or a hydrogel formulation. In one aspect, the induction regimen is completed prior to beginning administration of the maintenance regimen. In an alternative aspect, the maintenance regimen begins at or near the same time as the induction regimen.

In a particular embodiment, the induction regimen route of administration for the above methods is selected from intra-operative, intrathecal, intradiskal, peridiskal, epidural (including periradicular and transforaminal), or any combination thereof, and the maintenance regimen route of administration is selected from perispinal, IV, SC, intramuscular, and transdermal, or any combination thereof. In one aspect, the induction regimen is administered locally to an HD or site of SS (including within 10 cm of the HD or site of SS), and the maintenance regimen is administered systemically or parenterally.

In an embodiment, the induction regimen comprises a lower dose per administration to the subject than the maintenance regimen dose per administration.

In an embodiment, the direct above described methods of TNF-I or NFκB-I administration further comprise administering to the subject a therapeutically effective amount of a supplemental active ingredient (SAI). This SAI is selected from the group consisting of a second TAT, a corticosteroid, ozone, an antirheumatic drug, an LA, a neuroprotective agent, a salicylic acid acetate, a hydromorphone, a non-steroidal anti-inflammatory drug, a cox-2 inhibitor, an antidepressant, an anticonvulsant, a calcium channel blocker, and an antibiotic.

In an embodiment, the disclosure describes a method for improving the outcome of a spinal surgery procedure in a subject, wherein the subject meets at least one predetermined SOE for a spinal surgery procedure. This method comprises the following: a) optionally identifying the subject as a subject eligible for the spinal surgery procedure; b) administering to the subject a therapeutically effective amount of at least one direct TNF-I; and c) performing the spinal surgery procedure.

In an embodiment, also described herein is a method for improving the outcome of a spinal surgery procedure in a subject, wherein the subject meets at least one predetermined SOE for a spinal surgery procedure. This method comprises the following: a) optionally identifying the subject as a subject eligible for the spinal surgery procedure; b) administering to the subject a therapeutically effective amount of at least one NFκB-I; and c) performing the spinal surgery procedure.

In each of the above two methods, the subject may be diagnosed with HD and eligible for diskectomy; or diagnosed with SS and eligible for laminectomy. In one aspect, the above two methods include where the at least one predetermined SOE(s) for a spinal surgery procedure is selected from the following: a) a determination of eligibility of the subject for the spinal surgery procedure by a healthcare service provider (as evidenced by the following: i) a scheduling or request for scheduling by a healthcare service provider of the spinal surgery procedure for the subject; ii) a communication by a healthcare service provider to the subject that the subject has been determined to be eligible for the spinal surgery procedure; iii) a provision or offering by a healthcare service provider to the subject of a consent form for the spinal surgery procedure; iv) a receipt or execution by the subject of a consent form for the spinal surgery procedure, said consent form provided by the subject's healthcare provider; or v) a notation by the healthcare service provider in a tangible medium that the patient is eligible for the spinal surgery procedure); b) a determination of eligibility of the subject for the spinal surgery procedure by a qualified entity other than the subject's healthcare provider; c) the meeting by the subject of the eligibility criteria for a spinal surgery procedure in one or more CPG(s); d) eligibility of the subject for a diskectomy (as indicated by the subject meeting all of the following 3 clinical criteria: i) the subject exhibits symptoms of radiating back, neck, arm, and/or leg pain for a period of at least 4 to 8 weeks; ii) radiological (e.g., MR, CT, CT myelogram) determination of HD in the subject at the appropriate spinal location has been recorded; and iii) the subject exhibiting one or more of the following: evidence of spinal NR irritation or spinal cord deterioration (myelopathy) based on physical examination and/or electrodiagnostic studies; failure to respond adequately to one or more conventional non-invasive treatments; and limitation in the ability to perform normal activities such as walking, standing, or finding pain free positions); and e) eligibility of the subject for a laminectomy (as indicated by the subject meeting all of the following 3 clinical criteria: i) the subject exhibits symptoms of radiating back, neck, arm, and/or leg pain for a period of at least 8 weeks to 16 weeks; ii) a radiological (e.g., MR, CT, CT myelogram) determination of HD or SS in the subject at the appropriate spinal location has been recorded; and iii) the subject exhibits one or more of the following: evidence of spinal NR irritation or spinal cord deterioration (myelopathy) based on physical examination and/or electrodiagnostic studies; failure to respond adequately to one or more conventional non-invasive treatments; and limitation in the ability to perform normal activities such as walking, standing, or finding pain free positions).

In an embodiment, the method of administering a direct TNF-I includes administered a direct TNF-I in a time period that can be one or more of prior to, during, or after the time period of the spinal surgery procedure. Likewise, an NFκB-I may be administered in a time period that can be one or more of prior to, during, or after the time period of the spinal surgery procedure. In one aspect, the method for the administration of a direct TNF-I and an NFκB-I is according to a protocol that may be optionally interrupted for a time period prior to and/or after the spinal surgery procedure, after the invasive spinal surgery procedure.

In an embodiment, the methods include a scenario in which the therapeutic outcome of the subject from the spinal surgery procedure is improved and the improvement in therapeutic outcome includes at least one of the following: a) a reduction in one or more of the symptoms that rendered the subject eligible for the invasive procedure (where the one or more symptoms are selected from: i) the intensity or chronicity of the subject's radiating pain or radicular pain; ii) the degree of the subject's impaired ability to perform activities of daily living; and iii) the degree of the subject's neurologic impairment, muscle weakness, NR irritation); b) a reduction in the amount of a cytokine in the subject in a location of interest; c) an improvement in the abnormal findings previously observed on fluoroscopic or radiologic examination of the subject; d) the subject's no longer meeting the eligibility criteria in the predetermined SOE or CPG for the spinal surgery procedure; e) accelerated recovery of the subject from the spinal surgery procedure as evidenced by fewer days spent in the hospital in the post-operative period; f) an accelerated return of the subject to the activities of daily living; g) an increased quality of life of the subject; h) a decrease in the time to return to work for the subject (including a decrease in the time to restoration of functional capabilities for the subject); and j) a reduced incidence of failed procedure, as evidenced by a reduced incidence of eligibility for a repeat or revision spinal surgery procedure.

In an embodiment, the methods include a direct TNF-I selected from the group consisting of an antibody or antibody fragment, a fusion protein, a peptide, a SMIP, a small molecule, an oligonucleotide (such as an siRNA), an oligosaccharide, a soluble cytokine receptor or fragment thereof, a soluble TNF receptor Type I or a functional fragment thereof, a polypeptide that binds to TNF, and a dominant negative TNF molecule. Alternatively, the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

In an embodiment, the methods include an NFκB-I selected from the group consisting of sulfasalazine, sulindac, clonidine, helenalin, wedelolactone, pyrollidinedithiocarbamate (PDTC), IKK-2 inhibitors, and IKK inhibitors.

In an embodiment, the methods include an administration comprising: (a) an induction regimen comprising a direct TNF-I and (b) a maintenance regimen comprising a direct TNF-I.

In an embodiment, the administration comprises: (a) an induction regimen comprising an NFκB-I; and (b) a maintenance regimen comprising an NFκB-I.

Also described herein is a kit comprising a syringe, catheter, pump, or delivery device, where the syringe, catheter, pump or delivery device are adapted for epidural, intradiskal, or peridiskal administration, or any combination thereof, and a direct TNF-I. Alternatively, the kit may be adapted for epidural, intradiskal, or peridiskal administration, or any combination thereof, and an NFκB-I. In one aspect, the direct TNF-I is disposed within the syringe, catheter, pump, or delivery device, or is contained in a vial. In an alternative aspect, the NFκB-I is disposed within the syringe, catheter, pump, a delivery device, or delivery device, or is contained in a vial.

In an alternative embodiment, the aforementioned kit further comprises at least one SAI. In an aspect, the kit comprises an direct TNF-I is at a concentration in the range of from about 1 to about 100 mg/cc.

In an embodiment, the disclosure describes a pharmaceutical composition comprising a direct TNF-I at a concentration in the range of from about 1 to about 100 mg/cc, where the direct TNF-I is selected from adalimumab, CDP-870, and etanercept. In one aspect, the pharmaceutical composition further comprises an SAI. In another aspect, the pharmaceutical composition is disposed within a syringe, pump, catheter or delivery device.

Unless otherwise defined, all technical and scientific terms used herein have the meaning commonly understood by one of ordinary skill in the art to which this invention pertains. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the invention.

### BRIEF DESCRIPTIONS OF THE DRAWINGS

FIG. 1 demonstrates the ICs and IMs to which the TATs as described herein are directed.

FIG. 2 demonstrates the designated IC polypeptides TNF and IL-1 and the defined polypeptides of the TNF and IL-1 pathways.

FIG. 3 sets forth representative TNF-I doses for induction and maintenance regimens in pain patients using Humira® (adalimumab) or Enbrel® (etanercept).

FIG. 4 sets forth representative TNF-I doses for induction and maintenance regimens in pain patients using Remicade® (infliximab).

FIG. 5 sets forth representative TNF-I doses for induction and maintenance regimens in pain patients using Cimzia (certolizumab pegol, CDP870).

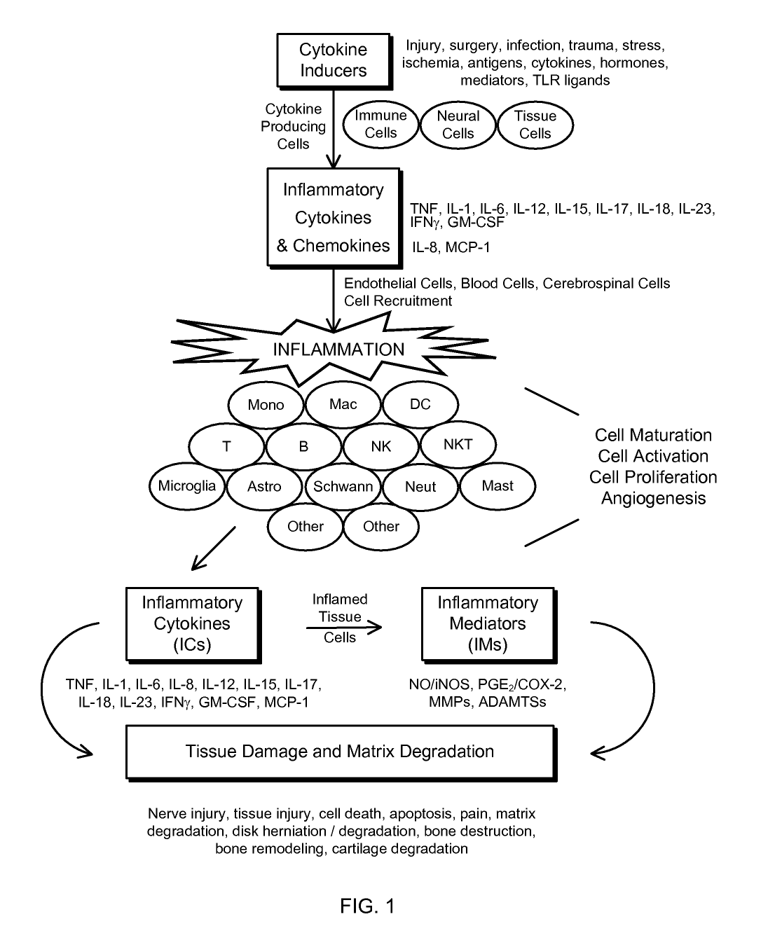

### DETAILED DESCRIPTION

### Definitions

Typically, and unless otherwise indicated, the term “spinal surgery procedure” and “spinal surgery” are used interchangeably and refer to an invasive spinal procedure that requires substantial removal of spinal tissues such as for example, all or part of one or more intervertebral disk(s), or all or part of one or more vertebra(e), including the lamina(e), without implantation of an implantable device, and without fusion of two or more vertebrae. Examples of such invasive spinal procedures without limitation include full or partial diskectomy and laminectomy, laminotomy, or laminoplasty. “Eligibility for a laminectomy” is used interchangeably with “Eligibility for a laminotomy” or “Eligilibility for a laminoplasty.” Repeat or revision embodiments of spinal surgery procedures, for example repeat diskectomy, are also included within the definition, provided they do not entail implantation of an implantable device, or fusion of two or more vertebrae.

As used herein, the term “other invasive spinal procedure” refers to an invasive spinal procedure that requires manipulation of spinal tissues, with minimal or no removal of spinal tissues, and also comprises neither implantation of an implantable device, nor fusion of two or more vertebrae. Examples of such invasive spinal procedures include adhesioloysis, radiofrequency neurotomy (RFN); and intradiskal electrothermal therapy (IDET). Repeat or revision embodiments of spinal surgery procedures, for example repeat adhesiolysis, RFN, or IDET, are also included within the definition, provided they do not entail implantation of an implantable device, or fusion of two or more vertebrae.

As used herein, the terms “tumor necrosis factor,” “tumor necrosis factor-alpha,” “TNF,” and “TNF-α” are used interchangeably to refer to a naturally occurring cytokine, which plays a key role in the inflammatory response, in the immune response and in the response to infection. The term “human TNF” (abbreviated as huTNF or hTNF), as used herein, is intended to refer to a human cytokine that exists as a 17 kiloDalton (kD) secreted form and a 26 kD membrane associated form, the biologically active forms of which are composed of trimers of noncovalently bound 17 kD or 26 kD molecules respectively.

As used herein, the term “inflammatory cytokine” is used interchangeably with “IC” and refers to one of the following designated polypeptides: TNF, IL-1, IL-6, IL-8, IL-12, IL-15, IL-17, IL-18, IL-23, IFN-γ, GM-CSF, MCP-1, IL-8 and MCP-1.

As used herein, the term “inflammatory mediator(s)” is used interchangeably with “IM” and refers to one of the following: MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, and PGE2.

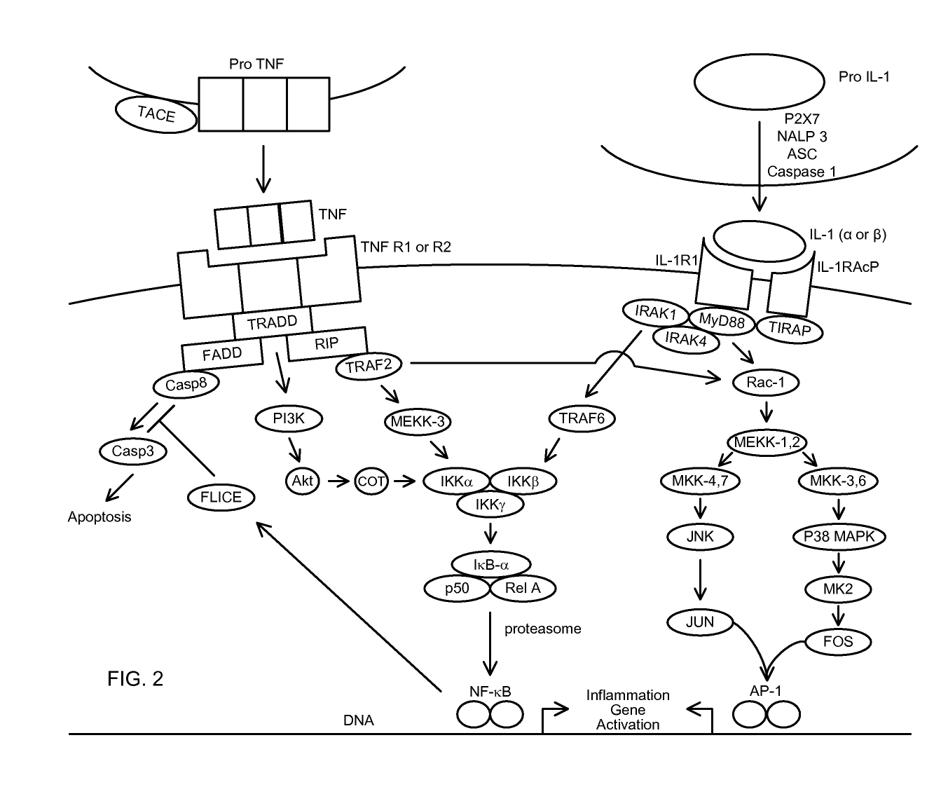

As used herein, the terms “inflammatory cytokine inhibitor” and “IC-I” are used interchangeably and refer to any molecule that blocks, suppresses or reduces gene expression, protein production and processing, protein release, and/or biological activity of: a) one of the following designated polypeptides: TNF, IL-1, IL-6, IL-12, IL-15, IL-17, IL-18, IL-23, IFNg, GM-CSF, and IL-8 (CXCR8) and MCP-1 (CCL2), or the designated polypeptide's biological receptor, coreceptor, or coligand, as described above, or b) one of the defined polypeptides within the designated polypeptide's pathway, as described above and described further below. See also, e.g., FIG. 2 for a depiction of the defined polypeptides in the TNF and IL-1 pathways.

An IC-I can be a “direct IC-I,” meaning a molecule (e.g., an antibody (Ab) or fusion polypeptide) that binds directly to and inhibits the biological activity of a designated polypeptide, its receptor, coreceptor, or coligand, or is a molecule (e.g., a nucleic acid such as an siRNA or antisense molecule) that binds directly to a nucleic acid molecule encoding the designated polypeptide or its receptor, coreceptor, or coligand and inhibits or reduces the expression of the designated polypeptide or its receptor, coreceptor, or coligand.

As used herein, the terms “inflammatory mediator inhibitor” and “IM-I” are used interchangeably and refer to any molecule that blocks, suppresses or reduces gene expression, protein production and processing, protein release, and/or biological activity of one of the following IMs: MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, and PGE2. An IM-I can be a “direct IM-I,” meaning a molecule (e.g., an Ab or fusion polypeptide or small molecule) that binds directly to and inhibits the biological activity of MMP-1 (collagenase-1), MMP-2 (Gelatinase A), MMP-3 (stromelysin), MMP-7 (Matrilysin), MMP-9 (gelatinase), MMP-13 (collagenase-3), ADAMTS4, ADAMTS5, iNOS, NO, COX-2, or PGE2, or meaning a molecule (e.g., a nucleic acid such as an siRNA or antisense molecule) that binds directly to a nucleic acid molecule encoding any of the foregoing IMs, inhibiting or reducing its expression.

Unless otherwise indicated, “small molecule,” and “small molecule inhibitor” are used interchangeably to refer to a molecule of low relative molecular mass that blocks, suppresses or reduces biological activity of a designated polypeptide. The term “low relative molecular mass” has art-recognized meaning, and refers to a molecule having a relative small number of atoms, typically less than 100 atoms (as compared to a protein, “biologic” or “macromolecule”). A small molecule can have a molecular weight of about 100 to 5000 daltons, e.g., about 500 to about 2000 daltons, or about 500 to about 1200 daltons.

As used herein, the terms “non-operative treatment” and “conventional non-invasive treatments” and “conservative care” are used interchangeably and mean one or more of watchful waiting by a healthcare provider, exercise, bed rest or reduced activity, physical therapy, administration of an NSAID, administration of a steroid, the use of an orthotic brace, and administration of oral analgesics including opioid analgesics.

As used herein, the term “peri-operative” means relating to, occurring in, or being the period around the time (e.g., before, during, and/or after) of a surgical operation.

“Interspinous route” refers to parenteral injection through the skin in the midline, in the interspace between two spinous processes, or via a paramedian approach, to deliver the therapeutic agent(s) in anatomic proximity to the spine.

“Intrathecal” means injection into the spinal canal (intrathecal space surrounding the spinal cord and intradural).

“Epidural” means in the space between the pia and dura mater, in which the nerve roots typically are found. “Periradicular” and “transforaminal” refer to specific types of epidural administration. “Periradicular” means within the epidural space, specifically in the region of the radicles (nerve roots). “Transforaminal” means through the vertebral foramen and within the epidural space, specifically in the region of the radicles. The terms “radicle,” “nerve root” and “NR” are used interchangeably.

“Intradiskal” means penetration of the outer wall and into the nucleus pulposus of a disk and/or into the annulus fibrosus of a disk.

“Peridiskal” means adjacent to an outer wall of the annulus fibrosus; outside but closely adjacent to an outer wall of the annulus fibrosus; and/or outside but closely adjacent to an endplate of an adjacent vertebral body.

“Perispinal” means in the paraspinal muscles.

“Intradiskal/epidural” means a combination of intradiskal, as defined above, and epidural, as defined above. For example, an “intradiskal/epidural” administration of a TAT could include administration of the TAT into the nucleus pulposus of a disk and administration of the TAT into the epidural space, e.g., using a needle adapted for intradiskal administration to administer the TAT intradiskally, followed by injection epidurally, either with the same or a different needle.

“Intradiskal/peridiskal” means a combination of intradiskal, as defined above, and peridiskal, as defined above. For example, an “intradiskal/peridiskal” administration of a TAT could include administration of the TAT into the nucleus pulposus of a disk and administration of the TAT into the peridiskal space adjacent to an outer wall of the annulus fibrosus, e.g., using a needle adapted for intradiskal administration to administer the TAT intradiskally, followed by injection peridiskally, either with the same or a different needle.

“Intradiskal/peridiskal/epidural” means a combination of intradiskal, peridiskal, and epidural, as defined above. For example, an “intradiskal/peridiskal/epidural” administration of a TAT could include administration of the TAT into the nucleus pulposus of a disk and administration of the TAT into the peridiskal space adjacent to an outer wall of the annulus fibrosus, and further administration of a TAT into the epidural space.

As used herein, an “induction regimen” has the following properties: it is administered by: 1) a more invasive route of administration than a maintenance regimen or more local site of administration than a maintenance regimen; and 2) a lower dose per administration than the dose per administration used in the maintenance regimen administered to the same subject, concurrent with or following the induction regimen.

As used herein, “treatment” means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the methods of the present invention.

A “therapeutically effective amount” is an amount sufficient to affect a beneficial or desired clinical result, such as prevention or treatment of injury and/or pain; the prevention, delaying, postponement, reduction, or elimination of the need for an invasive surgical procedure; or an improvement in the outcome of a subject that undergoes an invasive procedure.

As used herein, “delaying” or “postponing” are used interchangeably and mean to defer, hinder, slow, retard, and/or stabilize a subject's need for or eligibility for an invasive surgical procedure. This delay can be of varying lengths of time, depending on the history of the disease and/or individuals being treated. As is evident to one skilled in the art, a sufficient or significant delay can, in effect, encompass prevention, in that the individual does not need the procedure. A method that “delays” or “postpones” exhibition of the need for or the eligibility for the invasive procedure is a method that reduces probability of the need for or the eligibility for the procedure in a given time frame, when compared to not using the method. Such comparisons can be based on clinical studies, using a group of subjects sharing similar disease characteristics.

As used herein, a method for “improving the outcome” of an invasive procedure refers to a method that, for example, reduces severity or intensity of pain, symptoms, or disability, results in alleviation of one or more symptoms associated with the disease or disorder, reduces resting pain and/or mechanically-induced pain, shortens the duration of pain, symptoms, or disability, and/or reduces pain sensitivity or sensation, in a given time frame after the procedure when compared to the outcome observed when not using the recited method. Other examples of improved outcome are set forth further herein. Such comparisons can be based on clinical studies, using a group of subjects sharing similar disease characteristics.

As used herein, and unless otherwise indicated, the terms “patient,” “subject,” and “individual” are used interchangeably to refer to a vertebrate, and particularly a mammal including, without limitation, humans, farm animals, sport animals, pets, primates, horses, dogs, cats, mice and rats.

As used herein, the term “invasive,” when in the context of administration of a TAT, refers to the degree to which a particular administration regimen or mode of administration involves penetration of the delivery vehicle into the body, organ, or internal structures. A more invasive mode of administration refers to greater penetration into the body, organ, or internal structures than a less invasive mode. For example, a more invasive mode of administration can be evidenced through use of a longer needle, e.g., to penetrate further into the body, organ, or internal structures. Thus, intramuscular administration is more invasive than subcutaneous (SC) as the administration is deeper into the body. A more invasive mode of administration can be evidenced by the use of a catheter to administer into an internal organ, artery, or vein. A more invasive mode of administration can be evidenced by the requirement for local anesthesia during the procedure, e.g., to minimize accompanying pain directly due to the invasive procedure. A more invasive mode can be evidenced by a requirement for image guidance (e.g., ultrasound or radiographic imagery to guide the procedure) for the procedure (e.g., fluoroscopy for epidural or intradiskal administration). In some cases, a more invasive mode can involve greater risk, discomfort, or inconvenience to subject.

The following modes of administration are listed in order of invasiveness from highest to lowest: intra-operative, meaning into a surgical wound, to directly influence inflammation at the site of the surgical wound (e.g. into the wound in the region of the NR or disk); intradiskal; peridiskal, and intrathecal administration; epidural administration, including periradicular and transforaminal; intravenous (IV); perispinal and intramuscular; SC; and all other non-invasive modes of administration, including oral, intranasal, buccal, (including intrapulmonary and intrabronchial), and transdermal.

The term “pain” includes nociception and the sensation of pain, both of which can be assessed objectively and subjectively, using pain scores and other methods well-known in the art. Pain, as used herein, includes allodynia (i.e., increased response to a normally non-noxious stimulus) and hyperalgesia (i.e., increased response to a normally noxious or unpleasant stimulus), which can in turn, be thermal or mechanical (tactile) in nature. In some embodiments, pain is characterized by thermal sensitivity, mechanical sensitivity and/or resting pain. In other embodiments, pain comprises mechanically-induced pain or resting pain. In still other embodiments, the pain comprises resting pain. The pain can be primary or secondary pain, as is well-known in the art. Exemplary types of pain preventable or treatable by the methods of the present invention include, without limitation, back pain in the lumbar regions (low back pain) or cervical region (neck pain), leg pain, sciatic pain, radicular pain (experienced in the lower back and leg from lumber pathology, or in the neck and arm from cervical pathology), and neuropathic pain of the arm, neck, back, lower back, leg, and related pain distributions resulting from disk and spine pathology.

As used herein, “neuropathic pain” means pain arising from injury to the NR, dorsal route ganglion or peripheral nerve.

As used herein, “post-surgical pain” and “surgery-induced pain” are used interchangeably, and refer to pain arising in the recovery period of days or weeks following a spine surgical procedure. Specific examples of such pain that occur with increased frequency after spinal surgery include, without limitation, leg pain, back pain, neck pain, and/or arm pain. “Resting pain” refers to pain occurring even while the individual is at rest as opposed to, for example, pain occurring when the individual moves or is subjected to other mechanical stimuli. “Mechanically-induced pain” (interchangeably termed mechanosensory pain) refers to pain induced by a mechanical stimulus, such as the application of weight to a surface, tactile stimulus, and stimulation caused or associated with movement (including coughing, shifting of weight, etc.).

### I. Spinal Surgery Procedures

Invasive spinal procedures can be divided into two broad categories: 1) procedures that involve removal or manipulation of the damaged structure, without insertion of an indwelling device or fusion of the vertebrae, and 2) procedures that typically involve insertion of an indwelling device or fusion of the vertebrae. This invention pertains to the use of TATs to prevent or improve the outcome of the first category of spinal surgery procedures: those that involve removal or manipulation of the damaged structure, without insertion of an indwelling device or fusion of the vertebrae. These spinal surgery procedures include diskectomy (usually to treat HD) and laminectomy, laminotomy, and laminoplasty (usually to treat SS).

In diskectomy or laminectomy/laminotomy/laminoplasty procedures, pressure on a NR or the thecal sac is reduced by removing a compression. Standard invasive treatment for HD involves disk removal (diskectomy) to remove either the protruding portion of the damaged disk (partial diskectomy) or the entire disk (complete diskectomy), either with standard or minimal access percutaneous approaches. Diskectomy can be performed through posterior or anterior incison, or intrasdiskally either by mechanical, chemical, or thermal means.

Standard invasive treatment for SS involves laminectomies, laminotomies, and laminoplasties. Laminectomy removes the entire lamina. Laminotomy removes part of the lamina. Laminoplasty removes the ligamentum flavum, leaving the lamina otherwise intact or wedged open. In lumbar or sacral diskectomy procedures, a laminectomy is sometimes optionally performed to permit the removal or reshaping of a bulging or herniated lumbar or sacral disk.

### II. Methods for Identifying Subjects

As indicated previously, the inventor has discovered that patients who are suffering from moderate to severe disorders of the spine, such as HD or SS, and that are eligible for a spinal surgery procedure, such as a diskectomy or laminectomy, are candidates for treatment with TATs to prevent, delay, or improve the outcome of the invasive procedure. Such identification of this class of patients as eligible for treatment with a TAT is surprising in that the current standard of care posits that such patients will not benefit from administration of a currently approved TAT, such as the TNF-Is Enbrel® (etanercept), Humira® (adalimumab), and Remicade® (infliximab).

Accordingly, this disclosure provides a method of identifying a subject that could benefit therapeutically from administration of a TAT, such as a direct TNF inhibitor (direct TNF-I). The method includes determining that the subject meets at least one predetermined SOE for a spinal surgery procedure, thereby identifying the subject as one who could benefit.

Eligibility Criteria for Spine Surgical Procedures

The identification of a class of subjects as one that would benefit therapeutically from treatment with a TAT is based on the subject meeting the eligibility criteria in at least one (e.g., 1, 2, 3, 4, or more) of the predetermined SOE(s) for a spinal surgery procedure. Such SOEs, including CPGs, will change with changing healthcare practice and treatment options, and may vary from country to country. As SOEs including CPGs change, a skilled healthcare provider will be able to determine which patients are eligible for spinal surgery, relying upon professional judgement, upon CPGs generated by the provider's own healthcare organization, upon externally generated CPGs, and upon other guides to the current professional standard of care governing determination of eligibility for spinal surgery. A skilled healthcare provider will also be able to identify a currently relevant predetermined SOE, including a CPG. The predetermined SOEs including CPGs referenced herein are not meant to be all encompassing, nor will they remain static. They are illustrative of current predetermined SOEs and CPGs for spine surgical procedures.

A predetermined SOE could include, for example:
a) a determination of eligibility of the subject for the spinal surgery procedure by a healthcare service provider (e.g., a physician, physiatrist, osteopathic physician, physician's assistant, nurse practioner, physical therapist, nurse, or other qualified allied health professional), for example according to the healthcare provider's clinical judgement, or according to an internally or externally generated CPG by the healthcare organization in which the provider practices. Thus, the healthcare service provider has determined that the subject meets that provider's own criteria for undergoing the spinal surgery procedure, as evidenced by one or more of the following:
   i) a scheduling or request for scheduling by a healthcare service provider of the spinal surgery procedure for the subject. The fact that the procedure has been scheduled or requested for scheduling indicates that the healthcare service provider deems the subject to meet its criteria for undergoing the procedure;
   ii) a communication by a healthcare service provider to the subject that the subject has been determined to be eligible for the spinal surgery procedure. As above, the communication by the healthcare service provider indicates that the healthcare service provider deems the subject to meet its criteria for undergoing the procedure;
   iii) a provision to, or offering to the subject by a healthcare service provider of a consent form for the spinal surgery procedure. As above, the provision, offering, or receipt indicates that the provider deems the subject to meet its criteria for undergoing the procedure;
   iv) a receipt or execution by the subject of a consent form for the spinal surgery procedure, said consent form provided by the subject's healthcare provider. The fact that the subject has received and/or executed a consent form provided by the subject's healthcare provider indicates that the subject must be eligible for the procedure;
   v) a notation by the healthcare service provider in a tangible medium such as the patient's written or electronic medical record that the patient is eligible for the spinal surgery procedure. The fact that the provider has made such a notation of eligibility indicates that the subject must be eligible for the procedure.
b) a determination of eligibility of the subject for the spinal surgery procedure by a qualified entity other than the subject's healthcare provider, such as a healthcare provider organization [including a hospital, a health maintenance organization, a (HMO), a managed care organization, a defined healthcare provider network, or group practice], a national or local healthcare system, a hospital review committee, a professional guidelines committee, or a healthcare reimbursement agency, an insurance provider, or any other 3^{rd} party payor. The approval by one of the listed parties indicates that the subject meets a set of criteria set forth by the same to undergo the procedure, and is therefore eligible for the procedure;
c) the meeting by the subject of the eligibility criteria for a spine surgical procedure in one or more generally accepted CPG(s) governing eligibility for a spinal surgery procedure, generated by, for example: a healthcare service provider organization including a hospital, a health maintenance organization, a managed care organization, a group practice, or a defined healthcare provider network; a professional organization of healthcare providers such as, for example, North American Spine Society (NASS), American Academy of Orthopedic Surgeons (AAOS), or American Society of Interventional Pain Physicians (ASIPP); a healthcare reimbursement agency; a national or local healthcare system; a hospital review committee; a professional guidelines committee; or a 3^{rd} party payor. Representative examples, not intended to be limiting, of CPGs reflecting currently accepted standards of care for HD and SS include the CPG published by NASS on diagnosis and treatment of HD, which includes eligibility criteria for diskectomy surgery, and the NASS CPG on diagnosis and treatment of SS, which includes eligibility criteria for laminectomy surgery [3, 4].
d) the subject is eligible for a diskectomy, as indicated by the subject exhibiting:
   i) symptoms of radiating back, neck, arm, and/or leg pain for a period of at least 4 to 8 weeks;
   ii) a radiological (e.g., MR, CT, CT myelogram) determination of HD at the appropriate spinal location;
   iii) one or more of the following:
      a. evidence of spinal NR irritation or spinal cord deterioration (myelopathy) based on physical examination and/or electrodiagnostic studies;
      b. failure to respond adequately to one or more conventional non-invasive treatments;
      c. limitation in the ability to perform normal activities such as walking, standing, or finding pain free positions.
e) the subject is eligible for a laminectomy as indicated by the subject exhibiting:
   i) symptoms of radiating back, neck, arm, and/or leg pain for a period of at least 8 weeks to 16 weeks;
   ii) a radiological (e.g., MR, CT, CT myelogram) determination of HD or SS at the appropriate spinal location;
   iii) one or more of the following:
      aa. evidence of spinal NR irritation or spinal cord deterioration (myelopathy) based on physical examination and/or electrodiagnostic studies;
      bb. failure to respond adequately to one or more conventional non-invasive treatments;
      cc. limitation in the ability to perform normal activities such as walking, standing, or finding pain free positions.

Evidence of spinal NR irritation or NR inflammation can be determined by those having ordinary skill in the art. Representative findings would include: a) history suggestive of spinal NR irritation or spinal cord compression including the type and distribution of pain, especially the presence, absence, location and character of radiating pain, and a pattern of typical activities that either increase or decrease the painful symptoms; b) abnormal findings in a neurological examination, including abnormalities of gait or posture, motor or sensory loss in the distribution of an associated NR, or abnormal deep tendon reflexes; c) signs of NR irritation on physical exam, including evaluation of the subject's performance in a straight leg raise test, or in provocative maneuvers such as lateral side bending or forward bending.

Conventional non-invasive treatments for HD and SS typically include one or more of the following: bed rest, behavioral modification, physical therapy, administration of a course of non-steroidal anti-inflammatory agents, and administration of a course of analgesics, possibly including opioid agents. A subject can be considered to have failed a conventional non-invasive treatment if the subject's level of pain, injury, and/or disability is not significantly alleviated after a period of 4-8 weeks if the etiology is thought to be associated with HD [3], and 8-16 weeks if the etiology is thought to be due to SS [4].

In some cases, the generally accepted CPGs of a healthcare service provider for eligibility for the spinal surgery procedure can be the NASS CPGs for eligibility for the spinal surgery procedure, e.g., the NASS CPGs for treatment of HD [3], and for treatment of SS [4].

Once a subject has been identified, this identification can be further transmitted, e.g., to a healthcare service provider. The identification can also be memorialized, e.g., in a tangible medium of expression such as a patient's physical chart or record or a computer readable database. In some cases, the identification can be communicated to the subject, e.g., in the form of a recommendation that the subject undergo treatment with a TAT. In some cases, the subject will subsequently undergo treatment, e.g., administration of a TAT, according to any of the methods as disclosed further herein.

Therapeutic benefits to a subject can be determined and evaluated by those having ordinary skill in the art using known methods, e.g., the methods used to diagnose and/or determine eligibility for the spinal surgery procedure or the methods used to assess the effects of administration of a TAT as described herein, and can further include one or more of the following: objective or subjective measurements or assays of a reduction in pain, injury, or disability; an improved lifestyle; a delay, postponement, or reduction in need for a spinal surgery procedure; an improved outcome from surgery; a quicker return to work and/or function; improvement in standard measures of disability such as the Oswetry Disability Index, and improvement in accepted measures of improved social functioning, such as the Short Form 36.

### III. Methods for Preventing or Postponing a Spinal Surgery Procedure

Also provided herein are methods for treating a subject, e.g., preventing, reducing, delaying, eliminating, or postponing a subject's need for or eligibility for a spinal surgery procedure, where the subject meets at least one predetermined SOE for a spinal surgery procedure, for example by reducing the patient's pain or symptoms, so that the patient is no longer eligible for or no longer elects to undergo the invasive procedure.

The method includes: a) optionally identifying the subject as a subject eligible for the spinal surgery procedure, e.g., according to the methods described previously; and b) administering to the subject a therapeutically effective amount of at least one TAT, e.g., a direct TNF-I.

If a subject is optionally identified, then the identification can be further transmitted, e.g., to a healthcare service provider. The identification can also be memorialized, e.g., in a tangible medium of expression such as the patient's physical chart or record or a computer readable database. In some cases, the identification can be communicated to the subject, e.g., in the form of a recommendation that the subject undergo treatment with a TAT.

Any TAT including those as described more fully below can be employed in the methods. Any combination of TATs can be used in the methods, e.g., 2, 3, 4, or more TATs can be used in the method. Similarly, any administration regimen or route can be employed in the methods, including those described below.

In some cases, the effect of administering the TAT can be assessed to determine if the subject's eligibility for the spinal surgery procedure has been eliminated, prevented, delayed, reduced, or postponed. An assessment of the effect of an administration of TAT can be performed by methods known to those having ordinary skill in the art, such as the methods used to diagnose and/or determine eligibility for the spinal surgery procedure. Non-limiting examples of methods used to assess the effects of administration of a TAT can include:

a) determining the level or temporal duration of pain, degree of impaired mobility, or signs of spinal NR irritation in the subject as previously documented on physical examination, radiologic, or electrodiagnostic studies, compared to baseline characteristics;

b) determining the amount of a cytokine of interest, e.g., TNF (such as soluble TNF) in the subject (e.g., in a location of interest, such as a disk);

c) fluoroscopically or radiologically observing the subject (e.g., to evaluate the HD or SS); and

d) re-evaluating the history, physical exam, radiologic, and other criteria that rendered the patient eligible for the procedure, in order to determine whether the subject continues to meet the eligibility criteria in the predetermined SOE or CPG for the spinal surgery procedure.

Determining a level or duration of pain in a subject can be done using standard objective and subjective methods known to those having ordinary skill in the art, including methods employed to diagnose and/or determine eligibility for the spinal surgery procedure. Determining the amount of a cytokine of interest can also be performed using standard assays, such as bioassays, ELISA-based assays (e.g., ELISPOT assays), HPLC assays, and MS assays. Samples for measurement can be obtained from a location of interest, e.g., local to an HD or site of stenosis, including intradiskal biopsy samples.

Fluoroscopic or radiologic (e.g., MRI, X-ray, CT) observations can be performed using methods known to those having ordinary skill in the art. Typically the site observed will correlate with the location of the HD, SS or other spinal pathology.

In some case, the results of any of the assessment methods can be compared with a similar assessment performed prior to administration of the TAT. Multiple assessments during a course of TAT administration are also contemplated, e.g., 2, 3, 4, 5, 6 or more temporally separate assessments. Any suitable amount of time between assessments can occur, and can be determined by one having ordinary skill in the art. In some embodiments, from about 1 hour to about 2 months, or any time there between, elapses between assessments (e.g., 1 day, 2 days, 5 days, 1 week, 2 weeks, 4 weeks, 6 weeks, 8 weeks, or 2 months). Typically, one might expect to witness a response within about five half lives of a TAT or within about 2-8 weeks after initial administration of the TAT.

Administration of a TAT can include more than one administration of a TAT, e.g., at least 2, 3, 4, 5, 6 or more separate administrations of the TAT. The appropriate duration of time elapsed between the first and second (or any subsequent) administration of a TAT can be determined by one having ordinary skill in the art and may be determined based on the subject's need (e.g., pain level, responsiveness to the TAT, etc.), the route and regimen of administration; and an assessment of the effect of the first administration. Typically the time elapsed between administrations can range from about 1 day to about 2 months, or any time there between (e.g., 3 days, 5 days, 10 days, 20 days, 30 days, 45 days, 60 days). If a subject experiences a beneficial response from injection of a TAT, which has prolonged benefit (defined as one month or longer) and then experiences renewed symptoms anytime after this period, from 2 months to 20 or more years later, the administration of the TAT can be repeated in similar manner to the initial administration.

An administration of a TAT according to the methods described herein can treat the subject so that the subject does not undergo a spinal surgery procedure in the period following the TAT administration, ranging from the following 1-12 months (e.g., 1, 2, 3, 4, 5, 6, 7, 8, 9, 10, 11, or 12 months) to the following 1-20 years (e.g., 1, 2, 3, 4, 5, 6, 7, 8, 9, 10, 11, 12, 14, 16, 18, or 20 years) after the initial administration of the TAT. In some cases, the subject does not undergo the spinal surgery procedure, and thus the method has prevented or eliminated the need for the spinal surgery procedure.

### IV. Methods for Improvement of Outcome of Spinal Surgery Procedures

Any spinal surgery procedure, whether diagnostic or therapeutic, may disrupt and damage the disk and surrounding tissues. Such tissue disruption, by releasing ICs including TNF, can further inflame and damage the nearby NRs, peripheral nerves, and other adjacent tissues. Thus, spinal surgery procedures can inadvertently exacerbate as well as relieve a subject's symptoms and disability. Furthermore, spinal surgery procedures are not always successful in the long term. In some patients, while the spinal surgery procedure initially alleviates the subject's symptoms, the symptoms subsequently recur and/or progress, sometimes necessitating repeat surgery, typically with a less favorable likelihood of success.

The inventor has discovered that subjects who are eligible for and undergo a spinal surgery procedure, including those subjects who have been previously administered a TAT as described herein (e.g., to prevent, eliminate, postpone, delay, or reduce the need for the procedure), can also benefit therapeutically from administration of a TAT. Thus, in a patient in whom an initial TAT administration is performed to prevent, delay, or reduce the need for an invasive procedure, the initial administration or a repeat administration(s) can improve the therapeutic outcome if that patient eventually undergoes the procedure. In other cases, such an initial TAT administration may not be performed, but an administration of the TAT is coordinated to occur peri-operatively, e.g., at a time period prior to, during, and/or after the spinal surgery procedure, in order to improve the therapeutic outcome of the subject. In such situations, the provider may have determined that the subject was eligible for the procedure and may have decided to proceed with the procedure, and may elect to administer one or more administrations of a TAT peri-operatively. In yet other cases, both an initial TAT administration to prevent, delay, or reduce the need for the invasive procedure and a peri-operative administration are employed to improve the therapeutic outcome of a subject that does ultimately undergo the procedure.

Thus, in some embodiments, the present disclosure provides a method for improving a subject's outcome from a spinal surgery procedure, where the subject meets at least one predetermined SOE for a spinal surgery procedure. The method can include:

a) optionally identifying the subject as a subject eligible for the spinal surgery procedure;

b) administering to the subject a therapeutically effective amount of at least one TAT; and

c) performing the spinal surgery procedure.

Administration of the TAT can be performed using any route or regimen of administration, as described herein, including multiple administrations of one or more TATs. Administration of a TAT can be prior to, during, and/or after the spinal surgery procedure. The administration of a TAT prior to, during, and/or after the spinal surgery procedure can be in addition to an administration of a TAT completed prior to the spinal surgery procedure, e.g., an administration that delayed or postponed the spinal surgery procedure.

To address any perceived risk of increased infection risk upon administration of a TAT peri-operatively, the inventor has provided novel regimens of administration in which a TAT can either be administered locally, to reduce systemic exposure and infection risk, and/or can be optionally interrupted, e.g., for a time period prior to and/or after the spinal surgery procedure, with resumption of the TAT treatment regimen post-operatively. Peri-operative interruption of therapy would be at the discretion of the clinician responsible for managing the patient's therapy before, during, and/or after the spinal surgery procedure. The optional interruption time period prior to and/or after the spinal surgery procedure can be about equivalent or can be different. An optional interruption time period can range from about 1 day to about 14 days, or any time there between (e.g., 2, 4, 6, 8, 10, 12 days). In some embodiments, the optional interruption time period prior to and/or after the spinal surgery procedure is equivalent to about 1 to about 4 half-lives (t_{1/2}) (e.g., 1, 2, 3, or 4 half-lives) of the TAT in serum. Typically, the optional interruption period will be longer prior to the invasive procedure than after the invasive procedure.

The therapeutic outcome of the subject from the spinal surgery procedure can be improved, e.g., based on the administration of the TAT. An improvement in therapeutic outcome can be determined by methods known to those having ordinary skill in the art, including objective and subjective assessments, and can include at least one (e.g., 1, 2, 3, 4, 5, 6 or more) of the following:

a) a reduction in one or more of the symptoms that rendered the patient eligible for the invasive procedure, including a reduction in, for example:
i) the intensity or chronicity of the patient's radiating pain (e.g., radicular pain), including back, neck, leg or arm pain;
ii) the degree of the patient's impaired ability to perform activities of daily living, including moving, sitting, standing, bending, and working;
iii) the degree of the patient's neurologic impairment, muscle weakness, NR irritation, or other physical finding;

b) a reduction in the amount of a cytokine (e.g., soluble TNF) in the subject (e.g., in a location of interest);

c) an improvement in the abnormal findings previously observed on fluoroscopic or radiologic examination of the subject (e.g., an improved myelogram, MRI scan, CT scan, or other imaging exam);

d) the subject's no longer meeting the eligibility criteria in the predetermined SOE or CPG for the spinal surgery procedure;

e) accelerated recovery of the subject from the spinal surgery procedure, including fewer days spent in the hospital in the post-operative period;

f) an accelerated return of the subject to the activities of daily living;

g) an increased quality of life of the subject;

h) a decrease in the time to return to work for the subject;

i) a decrease in the time to restoration of functional capabilities for the subject; and

j) a reduced incidence of failed procedure, as evidenced by a reduced incidence of eligibility for a repeat or revision spinal surgery procedure.

### V. Other Invasive Spinal Procedures

The method of identifying and treating patients who would benefit from a TAT also surprisingly applies to patients eligible for other less invasive procedures for spinal pain not involving implantation of a spinal device or fusion of the vertebrae. Non-limiting examples of such procedures include percutaneous or endoscopic epidural adhesiolysis, RFN, or IDET. Typical spinal disorders that would make a patient eligible for one of these other invasive spinal procedures include NR entrapment, post-laminectomy syndrome or FBSS, facet joint syndrome, or DDD with internal derangement and associated diskogenic pain. For each of these procedures, current practice teaches away from offering a TAT to patients eligible for the invasive procedure.

Epidural adhesiolysis is used to treat patients with persistent pain following laminectomy or back surgery and sometimes for patients with persistent long-term back pain that has failed other conservative or non-surgical interventional pain procedures. The conditions treated with epidural adhesiolysis include epidural fibrosis and adhesive arachnoiditis which may result in NR entrapment or irritation. The latter conditions rarely occur in the absence of previous surgical and more often multiple surgical interventions while epidural fibrosis may rarely occur without previous surgery and accounts for some of the beneficial results seen with this technique in patients who have not had surgery. Post surgery, persistent symptoms can lead to a diagnosis of post-laminectomy syndrome, or failed back surgery syndrome (FBSS) which is the more usual criteria for a trial of epidural adhesiolysis.

Epidural adhesiolysis is a percutaneous invasive treatment for epidural fibrosis, scarring, adhesions, nerve entrapment syndrome, or post-laminectomy syndrome. Together, these conditions represent a significant cause of failed back surgery. In epidural adhesiolysis, a catheter is directed into the epidural space through the sacral hiatus, and hypertonic saline as well as physical manipulation of the catheter is used to break up adhesions and fibrosis in the epidural space that may have occurred as a result of surgery and may be a contributory cause to persistent pain following spinal surgery.

RFN is sometimes used to treat patients with persistent pain that has failed conservative and minimally invasive procedures such as corticosteroid injection and is often employed in the treatment of facet joint disease. Typically patients eligible for radiofrequency neurotomy of the nerves supplying the facet joint will demonstrate temporary relief with injection of LA into the joint, with or without steroids. In this case, radiofrequency lesion of the sensory nerve branches (medial branches) supplying the pathologic facet joint is sometimes employed to attempt to prolong the duration of benefit. Radiofrequency neurotomy or “RFN” employs a needle with a radiofrequency probe on its tip, which is directed under fluoroscopic guidance to selective NRs supplying facet joints, such as the medial branch. Using electrical stimulation, the probe allows confirmation that the tip is adjacent to sensory rather than motor branches of the NR. Radiofrequency induces a thermal injury to the sensory NR that selectively ablates sensory nerve function.

IDET is used to treat patients with diskogenic pain or other internal disk derangement conditions found in patients with moderate to severe DDD. IDET is sometimes performed for patients with diskogenic pain who demonstrate reproduction of symptoms with provocative diskography and have failed other more conservative non-interventional and interventional procedures. The goal of IDET is to extend the duration of symptom relief achieved with injection of LA and potentially to induce healing in a pathologic tear in the annulus of the disk and possibly to reduce the degree of herniation. IDET involves placement of a needle into the affected disk and threading of a thermal wire into the disk through the needle. The wire is inserted so as to localize near the suspected site of HD, either on the right or left side. An electrical current heats the wire, which results in a thermal injury to the disk contents and causes an inflammatory and ultimately fibrotic response to develop.

A subject can be determined to be eligible for epidural adhesiolysis, RFN, or IDET as follows:
a) the subject is eligible for adhesiolysis, as evidenced by the subject demonstrating the following [5]:
   i) persistent back pain of eight or more weeks duration that may be radiating; and
   ii) failure to respond to conservative treatment including trials of analgesics and/or fluoroscopically guided epidural or transforaminal injections; and
   iii) epidural lesions visualizable on epiduroscopy or a history of a spinal surgery procedure including either single or multiple laminectomy(s) or diskectomy(s), or other spinal surgery [6].
b) the subject is eligible for RFN, as evidenced by the subject demonstrating:
   i) facet joint pain of at least 8 weeks duration, typically originating in but not limited to the lumbar facets in the low back, in which case the pain may radiate into the buttock and proximal leg, and/or
   ii) facet joint pain manifesting as leg pain below the knee, that is lower in intensity than any pain, if present, that radiates to the buttock or thigh above the knee; and
   iii) temporary relief following at least two separate injections of two different LAs, with or without the addition of glucocorticoids, into the facet joint [6].
c) the patient is eligible for IDET, as evidenced by the patient demonstrating:
   i) midline back pain of greater than 6 weeks duration; and
   ii) failure to respond to conservative treatment including trials of analgesics and/or fluoroscopically guided epidural or transforaminal injections; and
   iii) diagnosis of diskogenic pain confirmed by provocative diskography; and
   iv) subject election to undergo IDET rather than an alternative spinal procedure; and optionally
   v) a characteristic history of increased pain with sitting, flexion, coughing, or sneezing [6].

### V. Targeted Anti-Inflammatory Therapies (TATs)

Structural Classes of TATs

TATs can be biologics (such as Abs, SMIPs, soluble receptor or coligands, or fusion proteins), polypeptides, nucleic acids, or small molecules.

Antibodies

In some embodiments of the invention, the TAT comprises an Ab, Ab fragment, or other functional equivalent thereof. Abs useful in the methods of the present invention include, without limitation, monoclonal Abs (mAbs), polyclonal Abs, Ab fragments (e.g., Fab, Fab′, F(ab′)2, Fv, Fc, etc.), chimeric Abs, mini-Abs or domain Abs (dAbs), dual specific Abs, bispecific Abs, heteroconjugate Abs, single chain Abs (SCA), single chain variable region fragments (ScFv), mutants thereof, fusion proteins comprising an Ab portion or multiple Ab portions, humanized Abs, fully human Abs, and any other modified configuration of the immunoglobulin (Ig) molecule that comprises an antigen recognition site of the required specificity, including glycosylation variants of Abs, amino acid sequence variants of Abs, and covalently modified Abs. Examples of dual specific Abs could include, but are not limited to, Abs directed to the following pairs of targets: two different antigens on the TNF molecule or TNF-R1 or R2; different chains of the TNF or TNF-R1 or R2 molecules; TNF and IL-1; TNF-R1 or R2 and TNF; TNF-R1 or R2 and IL-1; any antigen on TNF or TNF-R1 or R2 and any antigen on another IC such as IL-1, -6, -12, -15, -17, -18, -23, IFNg, GM-CSF, IL-8, MCP-1 (CCL2), and similar combinations. Methods for making such Abs are well known in the art. The Abs may be murine, rat, human, or any other origin (including chimeric, humanized, or fully human Abs). In one embodiment, the Ab recognizes one or more epitopes on an IC selected from TNF, IL-1, IL-6, IL-12, IL-15, IL-17, IL-18, IL-23, IFNg, GM-CSF, IL-8 and MCP-1 (CCL2), or recognizes one or more epitopes on an IM selected from MMP-1, 2, 3, 7, 9, 13, ADAMTS-4, 5, iNOS, NO, COX-2, and PGE2.

Antibodies also include, without limitation, agonist and antagonist Abs, as appropriate. As will be appreciated by those of skill in the art, binding affinities will vary widely between Abs, generally ranging from picomolar to micromolar levels. Methods for determining the binding affinity of an Ab are well known in the art. In some embodiments, the Ab binds an IC or IM and does not significantly bind the corresponding IC or IM from another mammalian species. In other embodiments, the Ab binds human TNF and optionally TNF from one or more non-human species.

In other embodiments, the Ab comprises a modified constant region, such as a constant region that is immunologically inert, e.g., does not trigger complement mediated lysis or stimulate Ab-dependent cell mediated cytotoxicity (ADCC) (see, e.g., U.S. Pat. No. 5,500,362). In other embodiments, the constant region is modified as described, for example, in [7]; PCT Application No. PCT/GB99/01441; and/or UK Patent Application No. 9809951.8.

Antibodies (e.g., human, humanized, mouse, chimeric) that can inhibit a protein's activity may be made by using immunogens that express the full length or a partial sequence of the protein (e.g., TNF), or cells that over expresses the protein. The Abs may be made by any method known in the art. The route and schedule of immunization of the host animal are generally in keeping with established and conventional techniques for Ab stimulation and production. Techniques for producing Abs are well known in the art including, without limitation, hybridomas, CHO cells, and other production systems; methods for primatizing or humanizing Abs and Ab fragments; methods for generating “fully human” Abs and Ab fragments; chimeric Abs; phage display technology; and recombinant technologies, such as transgenic animals and plants.

The Abs may be isolated and characterized using methods well known in the art. Abs may be isolated, for example, using conventional Ig purification procedures, such as ammonium sulfate precipitation, gel electrophoresis, dialysis, chromatography, and ultrafiltration.

SMIPs

A TAT can be a Small Modular Immuno-Pharmaceuticals (SMIP). SMIPs are single-chain polypeptides that are engineered to retain full binding and activity function of a monoclonal Ab (mAb); are approximately one-third to one-half the size of conventional therapeutic mAbs; and retain Fc-mediated effector functions. Examples of SMIP TATs for use in the present methods include TRU-015 and similar SMIPs that bind TNF or other ICs and IMs (Trubion Pharmaceuticals).

Soluble Receptors and Coligands

In some embodiments, the TAT comprises a soluble receptor or soluble co-ligand. The terms “soluble receptor”, “soluble cytokine receptor” (SCR) and “immunoadhesin” are used interchangeably to refer to soluble chimeric molecules comprising the extracellular domain of a receptor, e.g., a receptor of an IC or IM and an Ig sequence, which retains the binding specificity of the receptor and is capable of binding to the e.g., IC or IM (e.g., TNF). In one embodiment, a TNF SCR comprises a fusion of a TNF receptor amino acid sequence (or a portion thereof) from a TNF extracellular domain capable of binding TNF (in some embodiments, an amino acid sequence that substantially retains the binding specificity of the TNF receptor) and an Ig sequence. In some embodiments, the TNF receptor is a human TNF receptor sequence, and the fusion is with an Ig constant domain sequence. In other embodiments, the Ig constant domain sequence is an Ig heavy chain constant domain sequence. In other embodiments, the association of two TNF receptor-Ig heavy chain fusions (e.g., via covalent linkage by disulfide bond(s)) results in a homodimeric Ig-like structure. An Ig light chain can further be associated with one or both of the TNF receptor-Ig chimeras.

An example of a commercially available soluble receptor useful in the present invention is Enbrel® (etanercept). Enbrel® consists of recombinant human TNFR-p75-Fc fusion protein. The product is made by encoding the DNA of the soluble portion of human TNFR-p75 with the Fc portion of IgG.

Dominant-Negative Mutants

In other cases, a biologic TAT can be a dominant-negative mutant, e.g., of a polypeptide. One skilled in the art can prepare dominant-negative mutants of, e.g., the TNF receptor, such that the receptor will bind the TNF, thereby acting as a “sink” to capture TNF molecules. The dominant-negative mutant, however, will not have the normal bioactivity of the TNF receptor upon binding to TNF. The dominant negative mutant can be administered in protein form or in the form of an expression vector such that the dominant negative mutant, e.g., mutant TNF receptor, is expressed in vivo. The protein or expression vector can be administered using any means known in the art, such as intra-operatively, intraperitoneally, intravenously, intramuscularly, subcutaneously, intrathecally, intraventricularly, orally, enterally, parenterally, intranasally, dermally, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Antisense and siRNA Molecules

In another embodiment, a TAT may be an antisense or siRNA molecule, e.g., to a designated IC or one of the defined polypeptides in its pathway(s), or to an IM. Nucleotide sequences of the designated ICs and the defined polypeptides in their pathways, and of the IMs are known and are readily available from publicly available databases. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-O sugar modifications well know in the art.

In some embodiments, the TAT is a direct IC-I or a direct IM-I comprising at least one antisense or siRNA molecule capable of inhibiting or reducing the expression of a designated IC polypeptide, a defined polypeptide in the designated polypeptide's pathway, or an IM. Alternately, expression and/or release and/or receptor expression can be decreased using gene knockdown, morpholino oligonucleotides, RNA inhibition oligonucleotides (RNAi), or ribozymes, or any other methods that are well-known in the art.

Small Molecules

In some embodiments, the TAT comprises at least one small molecule IC-I or IM-I. The small molecule can be administered using any means known in the art, including via inhalation, intra-operative administration, intraperitoneally, intravenously, intramuscularly, subcutaneously, intrathecally, intradiskally, peridiskally, epidurally, perispinally, intraventricularly, orally, enterally, parenterally, intranasally, or dermally. In general, when the TAT is a small molecule, it will be administered at the rate of 0.1 to 300 mg/kg of the weight of the patient divided into one to three or more doses. For example, in an adult patient of normal weight, the doses may range from about 1 mg to about 5 g per dose.

An exemplary small molecule for use as a TAT in the present methods is thalidomide, which is an inhibitor of TNF production. The term “thalidomide” refers to an anti-inflammatory agent sold under the trademark THALOMID® (Celgene), and all pharmaceutically acceptable prodrugs, salts, solvate, clathrates and derivatives thereof. The term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but are not limited to, halogen, oxygen, nitrogen, sulfur, hydroxy, methoxy, alkyl, amine, amide, ketone, and aldehyde. Exemplary thalidomide derivatives include, without limitation, taglutimide, supidimide, compounds disclosed in WO 94/20085, 6-alkyl-2-[3′- or 4′-nitrophthalimido]-glutarimides and 6-alkyl-3-phenylglutarimides [see e.g., (8)]; and lenalidomide, a derivative of thalidomide sold under the trademark REVLIMID® (Celgene), also known as CC-5013, which is described, for example, in [9].

Other small molecules that possess TAT, particularly TNF-I, activity include, without limitation, tetracyclines (e.g., tetracycline, doxycycline, lymecycline, oxytetracycline, minocycline), chemically modified tetracyclines (e.g., dedimethylamino-tetracycline), hydroxamic acid compounds, carbocyclic acids and derivatives, lazaroids, pentoxifylline, napthopyrans, aminone, pimobendan, vesnarinone, phosphodiesterase inhibitors, and small molecule inhibitors of kinases. Small molecule kinase inhibitors include, without limitation, small molecule inhibitors of p38MAPK, COT, MK2, PI3K, IKKa,b,g, MEKK1,2,3, IRAK1,4 and Akt kinase. See also US Pat. Publications 2006/0046961; 2006/0046960; and 2006/0253100 for examples of small molecule inhibitors for use in the present methods.

Biogenerics, Biosimilars, Follow on Biologics, and Follow-on Proteins

The TAT, including a direct TNF-1, could also be a biosimilar, biogeneric, follow-on biologics, or follow-on protein version of a currently contemplated TAT, including a direct TNF-I. For example, once the patents covering Enbrel® (etanercept) expire, other manufacturers will likely produce molecules similar or identical to etanercept, by manufacturing processes that are substantially similar or the same, or different from, those used to manufacture Enbrel®. Their objective would be to make, offer to sell, and sell therapeutics similar or identical in structure and activity to Enbrel® (etanercept). Such molecules are generally referred to as biogenerics, generic biologics, biosimilars, follow on biologics, and follow on proteins, depending on details of the molecule, the manufacturing process and the regulatory pathway. In certain instances, the new product might differ by one or a few amino acids, which might be purported to improve the manufacturing efficiency or the therapeutic efficacy. In all such instances, these molecules are viewed as substantially the same as, or the same as currently contemplated TATs, including direct TNF-Is.

Targets and Examples of TATs

TATs for use in the invention can be IC-Is or IM-Is. In inflammation, each IC has a unique profile of biological activity, often representing multiple distinct activities. These activities are mediated by interaction of the cytokines with their receptors on a variety of inflammatory and tissue cell types. The cellular effects of ICs are mediated by intracellular signaling pathways, many of which result in activation of transcription factors which in turn activate transcription of genes encoding IC, proteinacious IM, and other proteins.

IC-Is

A TAT can be an inhibitor of one of the following IC designated polypeptides or one of the defined polypeptides in their pathways, as described further herein: TNF, IL-1, IL-6, IL-12, IL-15, IL-17, IL-18, IL-23, IFNg, GM-CSF, IL-8, MCP-1 (CCL2).

TNF-Is, including Direct TNF-Is

TNF is produced primarily by stimulated macrophages, T cells and mast cells by cleavage of Pro TNF by TNF alpha converting enzyme (TACE). TNF induces the production of IL-1, IL-6, IL-8, IL-17, GM-CSF, PGE₂ and NO from macrophages, thus placing TNF near the top of a proinflammatory cascade. TNF also induces the production of the matrix-degrading proteolytic enzymes, MMPs and ADAMTSs, from chondrocytes, fibroblasts and other cells.

The biological effects of TNF are mediated via binding of TNF to either of two receptors, TNFR1 and TNFR2. Several signaling pathways may be activated (FIG. 2). One pathway leads to NF-κB activation and is mediated by signaling proteins, including TRADD, RIP, TRAF2, MEKK-3, IKKα,β,γ, IκB-α, p50, Rel A and proteasomes. An alternative pathway to NFκB activation involves PI3K, Akt and COT prior to the IKK complex. Another pathway leads to apoptosis of the cell and is mediated by TRADD, FADD and Caspase-3 and 8 and blocked by FLICE. A fourth pathway leads to AP-1 activation and involves Rac-1, MEKK-1,2, MKK3,4,6,7, JNK, p38MAPK and MK2.

The term “TNF inhibitor” or “TNF-I” refers to any molecule which can block, suppress or reduce gene expression, protein production and processing, release, and/or biological activity of TNF, its biological receptor, coreceptor, or coligand, or a defined polypeptide in the TNF pathways (FIG. 2). Thus, examples of TNF-Is include inhibitors of any of the following polypeptides: ProTNF, TNF, TNFR1 and TNFR2, caspase 3, caspase 8, FADD, NFκB, IκB-alpha, TACE, TRADD, RIP, TRAF2, MEKK3, PI3K, Akt, COT, IKKalpha, IKKbeta, IKKgamma, p50, RelA, TRAF6, FLICE, Rac-1, MEKK-1,2, MKK3,4,6,7, JNK, p38MAPK, MK2, JUN and FOS.

A TNF-I can inhibit either or both of the two receptors TNFR1 (TNF receptor type 1) and TNFR2 (TNF receptor type 2). Some TNF-Is can inhibit a cysteine aspartase protease, such as caspase 3 or caspase 8; or can inhibit FADD; or can inhibit TRAF2. Some TNF-Is can inhibit IκB, a protein which inhibits the cell survival pathway mediator protein Nuclear factor-kappa B (NFκB). Some TNF-Is may inhibit NFκB. Examples of NFκB inhibitors include sulfasalazine, sulindac, clonidine, helenalin, wedelolactone, pyrollidinedithiocarbamate (PDTC), IKK-2 inhibitors, IKK inhibitors, and others, e.g., those set forth in US Pat. Publication 2006/0253100. Some TNF-Is may inhibit TNF converting enzyme (TACE), a metalloproteinase that processes pro-TNF into its mature, soluble form for release. Drugs that selectively inhibit TACE, and thereby effectively block the processing and release of mature TNF, show anti-inflammatory effects and significant decreases in cytokine production in vitro and in vivo.

Preferred inhibitors for use in the present methods are direct TNF-Is. Examples of direct TNF-Is useful in the practice of the present invention include, without limitation, the marketed products etanercept (Enbrel®, Amgen), infliximab (Remicade®, Johnson and Johnson), adalimumab (Humira®, Abbott Laboratories) and certolizumab pegol (Cimzia®; peg-antiTNF alpha Ab fragment) (formerly CDP 870; UCB/Celltech, now Nektar). Examples of direct TNF-Is currently in clinical development include the fully human anti-TNF mAb CNTO-148 (golimumab, Centocor/J&J), and the anti-TNF mAb AME-527 (Applied Molecular Evolution/Eli Lilly).

Examples of direct TNF-Is currently in pre-clinical development include the fully human anti-TNF mAb ABX-10131 (Abgenix/Amgen); several Ab fragments in development by companies such as Domantis/Peptech and AbLynx; and the SMIP TRU-015 being developed by Trubion Pharmaceuticals.

Other examples of direct TNF-Is include ABX-10131; polyclonal anti-TNF Abs such as made by therapeutic human polyclonals (THP); anti-TNF polyclonal anti-serum such as that made by Genzyme; pegylated soluble TNF receptor Type I (pegsunercept/PEGs TNF-R1); Onercept (recombinant TNF binding protein (r-TBP-1)); trimerized TNF antagonist; dominant negative TNF proteins such as Xencor's dominant negative TNF-I; modified sTNR1 (Biovation); Humicade® (CDP-570); and PN0621 (mini-Abs against TNF).

IL-1 Inhibitors, Including Direct IL-1 Inhibitors

IL-1 (a term which includes both IL-1α and IL-1β forms) is produced by processing of the precursor proteins, Pro IL-1α and Pro IL-1β, in an intracellular “inflammasome” involving P2×7, NALP3, ASC and Caspase-1 (FIG. 2). The predominant circulating form of IL-1 is IL-1β, whereas IL-1α primarily remains cell-membrane associated. IL-1 binds to its receptor, IL-1R1 and that complex then binds to IL-1RAcP (accessory protein), which enables signal transduction. The biological effects of IL-1 are mediated by two pathways (FIG. 2). One pathway leads to NF-kB activation and involves MyD88, TIRAP, IRAK1,4, TRAF6 and the IKK complex shared by the TNF pathway. The other pathway leads to AP-1 activation and links the MyD88/TIRAP/IRAK-1,4 complex with Rac-I and downstream elements shared by TNF.

The term “IL-1 inhibitor” or “IL-1-I” refers to any molecule which can block, suppress or reduce gene expression, protein production and processing, release, and/or biological activity of IL-1, its biological receptor, coreceptor, or coligand, or a defined polypeptide in the IL-1 pathways shown in FIG. 2. Examples of IL-1-Is include inhibitors of any of the following polypeptides: IL-1 alpha, IL-1 beta, Pro IL-1, P2×7, NALP3, ASC, Caspase-1, IL-IR1, IL-IRAcP, IRAK1, MyD88, TIRAP, IRAK4, TRAF6, Rac-1, MEKK-1, MEKK-2, MEKK-4, MEKK-7, JNK, JUN, FOS, MK2, p38 MAP kinase, MEKK-3, MEKK-6, AP-1, IKKalpha, -beta, or -gamma; IkB-alpha, p50, Rel A and NFκB.

Examples of IL-1-I are VX740 and VX765, small molecule caspase-I inhibitors previously in clinical development for rheumatoid arthritis (Vertex). Some IL-1-Is can inhibit p38 kinase (p38 MAP kinase). Over 100 p38 kinase inhibitors have been identified, many of which compete with ATP and are able to bind both active and inactive (phosphorylated and unphosphorylated) forms of the MAP kinase. In other cases, tyrosine-specific phosphatases can inhibit p38 MAPK by dephosphorylating the kinase at key positions. Treatment of arthritic animal models with synthetic p38 inhibitors suggests that p38 inhibition can produce protective anti-inflammatory effects in vivo. Small molecule inhibitors of p38 MAPK have demonstrated a broad range of anti-inflammatory effects mediated by changes in cytokine production. Exemplary small molecule p38 kinase inhibitors are described in US 2005/0025765.

A direct IL-1-I can be an inhibitor of an IL-1 receptor. Interleukin-1 receptor antagonist (IL-1 Ra) is a naturally occurring molecule which reduces the biologic effects of interleukin-1 by interfering with the binding of IL-1 to its receptor (IL-1 R1, interleukin-1 type 1 receptor). Kineret® (Amgen) is a recombinant form of IL-1 Ra which is FDA-approved for treating rheumatoid arthritis. Another example of a direct IL-1-I is AMG108, a mAb directed to IL-1R, currently in clinical development in rheumatoid arthritis (Amgen). AMG719 (sIL-1R2, Amgen), and IL-1 Trap (Regeneron), are also all direct inhibitors of IL-1. Another example of a direct IL-1-I is ACZ885 (a fully human anti-interleukin-1beta (anti-IL-1beta) mAb) in clinical development for Muckle-Wells Syndrome (Novartis).

IL-6 Inhibitors, Including Direct IL-6 Inhibitors

The effects of IL-6 are mediated by binding of IL-6 to IL-6Rα, either in soluble or membrane-bound form. The IL-6/IL-6Ra complex then binds to gp130 in the cell membrane to initiate signaling. Key proteins involved in the IL-6 pathway are JAK1, STAT1 and STAT3. The term “IL-6 inhibitor” or “IL-6-I” refers to any molecule which can block, suppress or reduce gene expression, protein production and processing, release, and/or biological activity of IL-6, its biological receptor, coreceptor, or coligand, or a defined polypeptide in the IL-6 pathway. Defined polypeptides in the IL-6 pathway are IL6Ralpha, gp130, JAK1, STAT1, and STAT3. An example of a direct IL-6-I is the humanized anti-IL6 receptor mAb Tocilizumab (Actemra®, Chugai). Another example of a direct IL-6-I is AMG 220, an Avimer™ protein, which binds to IL-6. AMG 220 is being studied in Crohn's disease patients. Another example of a direct IL-6-I is CNTO 328 (Anti IL-6 MAb) in clinical development for refractory multiple myeloma (Centocor). Another example of a direct IL-6-I is C326, an Avimer™ protein inhibitor of IL-6, in Crohn's Disease (Avidia).

IL-8 Inhibitors, Including Direct IL-8 Inhibitors

IL-8 is a chemokine also known as CXCL8. IL-8 mediates its activities through either of two receptors, CXCR1 and CXCR2, which are also receptors for other chemokines. Key proteins involved in the IL-8 pathway are PKC, PLC, PLD, Ras, rho and PI3K. The term “IL-8 inhibitor” or “IL-8-I” refers to any molecule which can block, suppress or reduce gene expression, protein production and processing, release, and/or biological activity of IL-8, its biological receptor, coreceptor, or coligand, or a defined polypeptide in the IL-8 pathway. Defined polypeptides in the IL-8 pathway are CXCR1, CXCR2, PKC, PLC, PLD, Ras rho and PI3K. An example of a direct IL-8-I is ABX-IL8, a fully human anti-IL-8 mAb previously in clinical development for psoriasis, COPD and chronic bronchitis (Abgenix).

IL-12 Inhibitors, Including Direct IL-12 Inhibitors

IL-12 is a heterodimer comprised of IL-12p40 and IL-12p35 chains, the former also being part of the IL-23 molecule. IL-12 mediates its activities through a heterdimeric receptor comprised of IL-12Rβ1 and IL-12Rβ2, again the former being part of the IL-23R. Key proteins involved in the IL-12 pathway include TYK2, JAK2 and STAT4. The term “IL-12 inhibitor” or “IL-12-I” refers to any molecule which can block, suppress or reduce gene expression, protein production and processing, release, and/or biological activity of IL-12, its biological receptor, coreceptor, or coligand, or a defined polypeptide in the IL-12 pathway. Defined polypeptides in the IL-12 pathway are IL-12p40, IL-12p35, IL-12Rβ1, IL-12Rβ2, TYK2, JAK2 and STAT4. An example of an IL-12-I is the small molecule STA-5326 Meslylate in clinical development to treat gut inflammation (Synta). An example of a direct IL-12-I is ABT-874, a human mAb directed against IL-12p40, in clinical development for psoriasis and other inflammatory diseases (Abbott). Another example of a direct IL-12-I is CNTO 1275 a human mAb directed against IL-12p40, in clinical development for psoriasis and other inflammatory diseases (Centocor).

IL-15 Inhibitors, Including Direct IL-15 Inhibitors

IL-15 mediates its activities by binding to a heterotrimeric receptor comprised of an IL-15Rα chain, an IL-2/15Rβ chain and the “common γ chain” γc. Key proteins involved in the IL-15 pathway include JAK1,3 and STAT3,5. The term “IL-15 inhibitor” or “IL-15-I” refers to any molecule which can block, suppress or reduce gene expression, protein production and processing, release, and/or biological activity of IL-15, its biological receptor, coreceptor, or coligand, or a defined polypeptide in the IL-15 pathway. Defined polypeptides involved in the IL-15 pathway are IL-15Ralpha, IL-2/IL-15Rbeta, the common gamma chain “gamma-c”, JAK1, JAK3, STAT3 and STAT5. An example of a direct IL-15-I is AMG 714, a fully human mAb (formerly called HuMAX15) directed against IL-15 in clinical development by Amgen/Genmab.

IL-17 Inhibitors, Including Direct IL-17 Inhibitors

IL-17 mediates its effects via an IL-17R that is expressed on virtually all cell types. Key proteins involved in the IL-17 pathway include TRAF6 and the same downstream IKK complex leading to NF-κB activation as in IL-1 pathway. The term “IL-17 inhibitor” or “IL-17-I” refers to any molecule which can block, suppress or reduce gene expression, protein production and processing, release, and/or biological activity of IL-17, its biological receptor, coreceptor, or coligand, or a defined polypeptide in the IL-17 pathway. Defined polypeptides in the IL-17 pathway are IL-17R, MyD88, TIRAP, IRAK1, IRAK4, TRAF6, IKKalpha, IKKbeta, IKKgamma, IkappaB-alpha, p50, Rel A, Proteasome, NFκB and FLICE.

IL-18 Inhibitors, Including Direct IL-18 Inhibitors

IL-18 binds to a 4-chain receptor complex comprised of IL-18Rα, IL-18Rβ, IL-IRAcP and a pathway chain. A naturally-occurring antagonist of IL-18 called IL-18BP blocks the binding of IL-18 to its receptor. Key proteins involved in the IL-18 pathway include MyD88 and all the downstream elements via TRAF6 leading to NF-κB activation as in IL-1 pathway. The term “IL-18 inhibitor” or “IL-18-I” refers to any molecule which can block, suppress or reduce gene expression, protein production and processing, release, and/or biological activity of IL-18, its biological receptor, coreceptor, or coligand, or a defined polypeptide in the IL-18 pathway. Defined polypeptides in the IL-18 pathway are Pro IL-18, P2×7, NALP3, ASC, Caspase-1, IL-18, IL-18Ralpha, IL-18Rbeta, IL-IRAcP, IL-18R signaling chain, IL-18BP, MyD88, TIRAP, IRAK1, IRAK4, TRAF6, IKKalpha, IKKbeta, IKKgamma, IkappaB-alpha, p50, Rel A, Proteasome, NFκB, FLICE, Rac-1, MEKK-1, MEKK-2, MKK3, MKK4, MKK6, MKK7, JNK, p38MAPK, MK2, JUN, FOS and AP-1.

IL-23 Inhibitors, Including Direct IL-23 Inhibitors

IL-23 is a heterodimer of IL-12p40 and IL-23p19 chains and binds to a heterodimeric IL-23 receptor comprised of IL-12Rβ1 and IL-23R. Key proteins involved in the IL-23 pathway include TYK2, JAK2 and STAT3. The term “IL-23 inhibitor” or “IL-23-I” refers to any molecule which can block, suppress or reduce gene expression, protein production and processing, release, and/or biological activity of IL-23, its biological receptor, coreceptor, or coligand, or a defined polypeptide in the IL-23 pathway. Defined polypeptides in the IL-23 pathway are IL-12p40, IL-23p19, IL-12Rβ1, IL-23R, TYK2, JAK2 and STAT3. An example of a direct IL-23-I is ABT-874, a human mAb directed against IL-12p40, in clinical development for psoriasis and other inflammatory diseases (Abbott). Another example of a direct IL-23-I is CNTO 1275, a human mAb directed against IL-112p40, in clinical development for psoriasis and other inflammatory diseases (Centocor).

IFNγ Inhibitors, Including Direct IFNγ Inhibitors

The effects of IFNγ are mediated by homodimers of IFNγ binding to a receptor comprised of an IFNγRα ligand-binding chain and an IFNγRβ signaling chain. Key proteins involved in the IFNγ pathway include JAK1, JAK2 and STAT1. The term “IFNγ inhibitor” or “IFNγ-I” refers to any molecule which can block, suppress or reduce gene expression, protein production and processing, release, and/or biological activity of IFNγ, its biological receptor, coreceptor, or coligand, or a defined polypeptide in the IFNγ pathway. Defined polypeptides in the IFNγ pathway are IFNγRα, IFNγRβ, JAK1, JAK2 and STAT3.

GM-CSF Inhibitors, Including Direct GM-CSF Inhibitors

GM-CSF binds to a heterodimeric receptor comprised of GMRα and a common β subunit, βc. Key proteins involved in the GM-CSF pathway include JAK2, STAT5, SHP-2, RAS and Raf-1. The term “GM-CSF inhibitor” or “GM-CSF-I” refers to any molecule which can block, suppress or reduce gene expression, protein production and processing, release, and/or biological activity of GM-CSF, its biological receptor, coreceptor, or coligand, or a defined polypeptide in the GM-CSF pathway. Defined polypeptides in the GM-CSF pathway are GMRalpha/Beta-c, JAK2, STAT5, SHP-2, RAS and Raf-1.

MCP-1 Inhibitors, Including Direct MCP-1 Inhibitors

MCP-1 is a chemokine also known as CCL2. MCP-1 mediates its activities by binding to a single receptor, CCR2. Key proteins involved in the MCP-1 pathway include PKC and the same IKK complex and downstream elements as in TNF/IL-1 pathway leading to NF-kB activation. The term “MCP-1 inhibitor” or “MCP-1-I” refers to any molecule which can block, suppress or reduce gene expression, protein production and processing, release, and/or biological activity of MCP-1, its biological receptor, coreceptor, or coligand, or a defined polypeptide in the MCP-1 pathway. Defined polypeptides in the MCP-1 pathway are CCR2, PKC, IKKalpha, IKKbeta, IKKgamma, IkappaB-alpha, p50, Rel A, Proteasome, NFκB and FLICE. An example of a direct MCP-1I is ID9, a mAb directed against the MCP-1 receptor CCR2 (Millenium).

IM-Is

A TAT can be an inhibitor of one of the following IMs: MMP-1,2,3,7,9,13; ADAMTS-4, 5; iNOS, NO, COX-2, and PGE2.

MMP Inhibitors, Including Direct MMP Inhibitors

The term “MMP-1, 2, 3, 7, 9, 13 inhibitor” or “MMP-1-I, 2-I, 3-I, 7-I, 9-I, 13-I” refers to any molecule which can block, suppress or reduce gene expression, protein production and processing, release, and/or biological activity of the respective MMP-1, 2, 3, 7, 9, or 13 polypeptide, or the biological receptor, coreceptor, or coligand of the same. Examples of broad-spectrum (nonspecific) direct MMP-Is include the small molecule compounds marimastat and batimastat, previously in clinical development (British Biotech, Inc).

An example of a class of direct MMP-13-I with selectivity relative to other MMPs is the small molecule genus of 3-hydroxy-4-arylsulfonyltetrahydropyranyl-3-hydroxamic acids previously in clinical development (Pfizer).

An example of a direct MMP-2-I and direct MMP-9-I is XL784, a relatively selective small molecule compound in clinical development (Exelixis).

iNOS Inhibitors, Including Direct iNOS Inhibitors

The term “iNOS inhibitor” or “iNOS-I” refers to any molecule which can block, suppress or reduce gene expression, protein production and processing, release, and/or biological activity of iNOS, or its biological receptor, coreceptor, or coligand. An example of a direct iNOS-I is GW274150, a small molecule compound in clinical development for rheumatoid arthritis and migraine (GSK). Another example of a direct iNOS-I is aminoguanidine, a small molecule compound evaluated in clinical endotoxemia (Radboud University). Another example of a direct iNOS-I is SC-51, a small molecule compound in clinical development for asthma (Pfizer).

COX-2 Inhibitors, Including Direct COX-2 Inhibitors

The term “COX-2 inhibitor” or “COX-2-I” refers to any molecule which can block, suppress or reduce gene expression, protein production and processing, release, and/or biological activity of COX-2, or its biological receptor, coreceptor, or coligand. Examples of direct COX-2-I are celecoxib (Celebrex®, Pfizer) and rofecoxib (Vioxx®, Merck), small molecule compounds for treatment of inflammation and pain.

Combination Therapies

Multiple TAT Inhibitors, Including Multiple TNF-I

The present disclosure also contemplates the use of multiple TATs in the methods described herein. The combination of different TATs that have specificity for different points in a pathway, e.g., a TNF pathway, or different points in two or more different pathways, may be more efficient than the use of a single TAT. For instance, TNF itself may be inhibited at multiple points and by targeting various mechanisms in the TNF pathways. Potential inhibition points include TNF transcriptional synthesis, translation, or shedding mediated by MMPs. TNF and other similar bioactive substances are first produced in an inactive form and transported to the cell membrane. Upon activation, the active part of the pro-TNF is cleaved and released. This process is called shedding and may be initiated by one or more MPs. TNF may also be inhibited after its release, either by Abs (e.g., by infliximab, adalimumab, or CDP-870) or soluble receptors (e.g. etanercept).

The combination of two or more drugs that act through different mechanisms may therefore induce a more efficient inhibition of an IC or IM pathway than the use of one single drug. In one embodiment, a direct TNF-I is used in combination with a second direct TNF-I, or with a non-specific TNF-I or an inhibitor of a different IC or IM. In another embodiment, a direct TNF-I is used in combination with an NFκB inhibitor such as sulfasalazine, sulindac, clonidine, helenalin, wedelolactone, pyrollidinedithiocarbamate (PDTC), IKK-2 inhibitors, IKK inhibitors, and others, e.g., those set forth in US Pat. Publication 2006/0253100.

Supplemental Active Ingredients

A TAT, e.g., TNF-I, may be administered in combination with other drugs or compounds, provided that these other drugs or compounds do not significantly reduce or eliminate the desired results according to the present invention, e.g., the effect on a IC or IM of interest such as TNF. Specific methods of the invention comprise administering a TAT in combination with an SAI. The SAI can be any TAT. Further, the SAI can be any therapeutic agent capable, for example, of relieving pain, providing a sedative effect or an antineuralgic effect, or ensuring patient comfort. Examples of the SAIs include, but are not limited to, opioid analgesics, non-narcotic analgesics, anti-inflammatories, cox-2 inhibitors, α-adrenergic receptor agonists or antagonists, ketamine, anesthetic agents, NMDA antagonists, immunomodulatory agents, immunosuppressive agents, antidepressants, anticonvulsants, antihypertensives, anxiolytics, calcium channel blockers, muscle relaxants, corticosteroids, hyperbaric oxygen, neuroprotectants, antibiotics, other therapeutics known to relieve pain, and pharmaceutically acceptable salts, solvates, hydrates, stereoisomers, clathrates, prodrugs and pharmacologically active metabolites of any of the foregoing therapeutic agents.

In another embodiment, the supplement active ingredient is a non-steroidal anti-inflammatory drug (NSAID), corticosteroid, slow acting antirheumatic drug (SAIRD), disease modifying antirheumatic drug (DMARD), short-acting LA, or long-acting LA. In yet another embodiment, the SAI is a propionic acid derivative, such as ibuprofen or naproxen. Structurally related propionic acid derivatives having similar analgesic and anti-inflammatory properties are also intended to be encompassed by this group. In another embodiment, the SAI is an acetic acid derivative, for example alclofenac, diclofenac sodium, or sulindac. Structurally related acetic acid derivatives having similar analgesic and anti-inflammatory properties are also intended to be encompassed by this group. The SAI may also be a fenamic acid derivative such as, without limitation, enfenamic acid, etofenamate, or flufenamic acid. Structurally related fenamic acid derivatives having similar analgesic and anti-inflammatory properties are also intended to be encompassed by this group.

In other embodiments, the SAI is a carboxylic acid derivative, a butyric acid derivative, or oxicam, a pyrazole, or a pyrazolon. In another embodiment, the SAI is an antibiotic. Exemplary antibiotics include, without limitation, sulfa drugs (e.g., sulfanilamide), folic acid analogs (e.g., trimethoprim), beta-lactams (e.g., penacillin, cephalosporins), aminoglycosides (e.g., stretomycin, kanamycin, neomycin, gentamycin), tetracyclines (e.g., chlorotetracycline, oxytetracycline, and doxycycline), macrolides (e.g., erythromycin, azithromycin, and clarithromycin), lincosamides (e.g., clindamycin), streptogramins (e.g., quinupristin and dalfopristin), fluoroquinolones (e.g., ciprofloxacin, levofloxacin, and moxifloxacin), polypeptides (e.g., polymixins), rifampin, mupirocin, cycloserine, aminocyclitol (e.g., spectinomycin), glycopeptides (e.g., vancomycin), and oxazolidinones (e.g., linezolid).

In another embodiment, the SAI is capable of providing a neuroprotective effect. In addition to TNF, other examples of neuroprotective agents include, without limitation, erythropoietin (Epo), Epo derivatives or mimetics, and other compounds that stabilize or protect neurons from injury. Epo and its derivatives or mimetics might offer particular advantages, or otherwise be particularly appropriate, to patients undergoing surgery. Usage of Epo or Epo-mimetics as neuroprotectants may be limited by the difficulty in separating the neuroprotective effects of Epo from the erythrogenic effects. However, a particular setting in which such erythrogenic “side effects” are acceptable is in patients about to undergo surgery, in whom a moderate and temporary increase in hematocrit may be desirable. Thus, in peri-operative usage to improve surgical outcome, Epo may offer surprising advantages as a neuroprotectant.

The SAI could also be ozone as delivered to the spinal structure by ozone therapy [10].

### VII. Administration Regimens

Any route of administration for a TAT and any type of formulation of a TAT can be used in the present methods. Routes of administration for currently approved TATs, such as TNF-Is, are known to those of ordinary skill in the art, consisting primarily of systemic injection, e.g., intramuscular injection, SC injection, or IV infusion [11]. Other more invasive routes of administration, however, are also specifically contemplated in the present methods, e.g., including intrathecal, intradiskal, and epidural routes. Thus, a TAT can be administered using any of the following routes of administration: intra-operatively, intravenously, intramuscularly, SC, intrathecally, intradiskally, peridiskally, epidurally, perispinally, orally, enterally, parenterally, intranasally, dermally (e.g., transdermally), or by inhalation.

A TAT composition can be administered to a site, e.g., a site of a spinal surgery procedure, using any suitable method, such as delivery through a needle or other cannulated device (see, e.g., U.S. Pat. Nos. 6,375,659, 6,348,055 and 6,582,439). The TAT composition may be delivered via a single injection, or by multiple injections at or near the surgical site. A suitable volume of a TAT composition can be determined using methods well known in the art, for example by adding barium, tungsten, or other substances to render the material radiopaque.

In preferred embodiments of the present invention, a pump is used to deliver one or more TATs and optionally other therapeutic agents continuously over an extended period of time, or intermittently at distinct times of administration. These pump devices preferably comprise a pump; a reservoir coupled to the pump; and a catheter operably coupled to the pump and configured to deliver the therapeutic agent to the target site. For purposes of allowing ease of treatment over an extended period of time, the catheter may be designed such that it is removable from the pump, and may be capped and retained within the patient's body such that repeated doses may be administered through the catheter without the need for repeatedly inserting and removing the catheter. The timing and dosage regimen may be pre-set, may be monitored and adjusted by computer, or may be monitored and adjusted by the patient or a treating care worker to provide the appropriate dosage at the right time. Use of such pump and catheter systems is particularly advantageous for allowing administration of the maintenance dosage regimen of TATs in accordance with the present invention. The catheter may be implanted at the time of a spinal surgery procedure, such as a diskectomy, such that subsequent dosage and targeting of TATs to the particularly affected areas may be accomplished without further surgical intervention. A pump can be an infusion pump, an osmotic pump, or an interbody pump.

In some embodiments, a controlled release formulation, e.g., a depot, is used to deliver one or more TATs. A controlled release formulation can include, without limitation, a capsule, microsphere, particle, gel, wafer, pill, etc. A controlled release formulation can exhibit a controlled release rate of the one or more TATs, e.g., over a period from about 12 hours to about 3 months, or any time therebetween, e.g., 1 day to 1 week; 1 day to 1 month; 1 day to 2 months; etc. A controlled release formulation can include one or more biopolymers known to those having ordinary skill in the art, e.g., poly(alpha-hydroxy) acids, poly(lactide-co-glycolide) (PLGA), polylactide (PLA), polyglycolide (PG), PEG, PEG derivatives, PEG conjugates, polyvinyl alcohol (PVA), polyurethane esters, polycarbonates, copolymers, and others, including those as set forth in US 2006/0046961.

Administration of a TAT can be local and/or targeted or non-local; through more invasive or less invasive means; and at any suitable dose, e.g., as determined by a healthcare service provider.

Local and/or Targeted Administration

As described herein, the methods can utilize local or targeted administration of a TAT. Although these methods of administration can be moderately invasive, they are less invasive than a surgical procedure, and the local and/or directed administration of the TAT may offer the best way to selectively address the particular injury to the spine, disk, or surrounding nerves. For example, the induction regimens of the present invention can involve locally directed administration of one or more TATs to allow effective interruption of the inflammatory pathways, e.g., the TNF pathway, and to alleviate neuropathic pain. Local administration may also reduce unwanted systemic side effects of the TAT, by permitting the use of lower doses, or by limiting systemic exposure through local delivery. In some embodiments, local administration can mean placement of the delivery vehicle within 10 cm (e.g., within 10, 9, 8, 7, 6, 5, 4, 3, 2, 1, or 0.5 cm) of the site of the presumed injury, into an appropriate location in a suitable form. In some cases, local administration permits the delivery of higher TAT concentrations than could be achieved systemically.

Intrathecal Delivery

One means of local delivery is the use of an intrathecal delivery system. Intrathecal delivery systems frequently comprise an infusion pump and an intraspinal catheter. One or more TATs may thus be delivered to the spinal canal or intrathecal space. Both the pump and the catheter may be implanted. In such cases, the pump is usually programmable, such that chronic infusion of one or more TATs may be accomplished over a period of time, and the pump or a reservoir may be periodically refilled. Alternatively, the pump may be external, and used for the delivery of one or more TATs. In such cases, administration may be controlled manually, or a programmable pump may still be used. Intrathecal delivery may be the preferred means of systemic delivery because the drug does not enter the bloodstream, and thus will not cross the blood-brain barrier into the brain.

Intrathecal or Epidural Pump and Catheter Systems

In the present invention, one or more TATs may be administered by means of an intrathecal or epidural delivery device. Such a delivery device can be any one of the currently marketed Medtronic Sofamor Danek intrathecal drug delivery devices, including but not limited to any of the SynchroMed®EL models, any of the SynchroMed®II models, or the MiniMed Paradigm®-REAL-TIME Insulin Pump and Sertable™ infusion sets, described above, and/or their successors. These pumps may be implanted to prevent or postpone the need for a spinal surgery procedure, and/or to improve the outcome of the spinal surgery procedure, e.g., to treat surgery-induced injury and pain using one or more TATs. Additionally, one or more TATs may be used to coat the intrathecal catheter prior to implantation. One or more TATs may also be used in the patient screening test in order to assess the effectiveness of the drug prior to implantation of an intrathecal delivery device.

A number of infusion pumps are currently marketed by Medtronic Sofamor Danek, known collectively as the SynchroMed® Infusion system. The Medtronic SynchroMed® Programmable Drug Infusion Pump is a fully implantable, programmable, battery-powered device that stores and delivers medication according to instructions received from the programmer.

Intrathecal drug delivery provides a treatment option that is fully reversible, i.e., the system can be turned off or fully removed with little or no consequence. A further benefit of this system is that in most patients, pain is alleviated using a lower dose of medication than is required to achieve the same effect via oral or IV routes because the pain medication is delivered directly to the appropriate (e.g., intrathecal or epidural) space. For example, pain relief can be achieved using intrathecal drug delivery with a dose that is 99.9967% lower compared to the dose required to achieve the same result orally [12]. This also reduces the side effects that may be associated with higher doses of the medication, such as nausea, vomiting, sedation, and constipation, thus improving the patient's quality of life. Unlike long-term IV or epidural therapy, intrathecal drug delivery also allows a patient to tailor his medication to his lifestyle. Under the guidance of their clinician, patients can administer themselves an additional dose of medication, known as a “bolus” dose, if they feel a spike in pain or in preparation for an activity that is expected to result in a spike in pain. Finally, as the system is fully implanted, there is a low risk of infection.

Epidural, Intradiskal, and Peridiskal Administration

Administration using an epidural syringe is a well-known method of administering therapeutic agents, such as anesthetics or steroids, to the spine. Using fluoroscopic or other means to guide the epidural syringe to the desired location, therapeutics may be delivered to the area known as the epidural space, which is adjacent to the dura mater and within the spinal canal formed by the surrounding vertebrae. By administering TATs using an epidural syringe, a single dose may be targeted directly to the area of insult or injury near the spine. Alternatively, use of an epidural catheter and pump system allows for an extended dosage regimen of repeat dosings to the epidural space.

A particularly useful means for administering TATs for an induction regimen as described herein comprises intradiskal administration. In preferred embodiments, intradiskal administration is accomplished using devices such as intradiskal or epidural syringes and other spinal injections, optionally combined with fluoroscopic guidance to provide means for conducting diskography for targeting TATs to the damaged disk or disks. In one particular embodiment, prior to or subsequent to intradiskal injection of one or more TATs, one or more TATs may additionally be administered targeted to the area just adjacent to the disk (the peridiskal area) and/or epidurally. Thus, in certain preferred embodiments of the invention, a single epidural syringe, or other means of spinal injections, may be used to administer one or more TATs, with or without other active agents, such as an LA, steroids, or other treatment, both intradiskally and peridiskally and/or epidurally (“intradiskal/peridiskal administration” or “intradiskal/epidural administration” or “intradiskal/peridiskal/epidural administration”). In one embodiment, the syringe may have two compartments, each containing a dose of at least one TAT intended for its respective targeted area. In another embodiment, the surgeon administering the TATs can manipulate the syringe in a manner such that part of the dosage is injected intradiskally, for example, by depressing the syringe lever only partway, thereby administering an intradiskal dosage; and retaining a peridiskal/epidural dosage to remain in the syringe; while the syringe is being withdrawn from the disk, the surgeon can administer the peridiskal/epidural dosage to the peridiskal/epidural region by pausing while the needle is adjacent to, but outside of the affected disk, and depressing the syringe further to administer the peridiskal/epidural dosage. Analogously, the surgeon can pause during insertion of the syringe and, while the needle is located adjacent to, but has not yet pierced, the affected disk, depress the syringe lever partway in order to direct a peridiskal/epidural dosage to the peridiskal/epidural area. Following such administration, the syringe can then be guided into the disk, and an intradiskal dosage administered.

Intradiskal administration can also be combined with other therapies, such as IDET, or with a diagnostic apparatus, such as the pump used for functional anesthetic diskograpy owned by Kyphon.

Other Means of Local and Targeted Administration

Other means of local administration include PR infiltration under fluoroscopic guidance, implants which are coated with a substance comprising one or more TATs, or biomaterials which comprise one or more TATs, and which are designed for the controlled delivery of TATs, including bioresorbable materials, e.g., controlled release formulations as described above which will release the TATs as they are resorbed into the body. Suitable resorbable materials are well known to those having ordinary skill in the art.

Systemic, Non-Local, and/or Non-Targeted Administration

In addition to local or targeted administration, the methods and materials of the present invention may also utilize systemic administration of one or more TATs. Unlike local or targeted administration, systemic administration tends to be less invasive, is typically “non-local” to the site of injury, and, importantly, may be performed as an out-patient treatment, or may even be self-administered by the patient. Thus, the systemic means of administration are advantageous in that they are less disruptive to the patient's life, and therefore, may result in improved compliance by patients with the prescribed regimens.

Systemic administration of one or more TATs can be used in any regimen, and is frequently used for the maintenance regimen in an induction-maintenance regimen as described herein. The maintenance regimens may provide for long-term relief of back pain or neuropathic pain by administering one or more TATs to allow the continued inhibition of the inflammatory pathway(s).

Parenteral Administration

Parenteral administration includes various methods of infusion or injection of the drug. Preferred methods of parenteral administration may include IV injection or infusion directly into the bloodstream. Other methods of parenteral administration include intramuscular; SC; transdermal; and intraperitoneal administration.

Other Means of Systemic Delivery

Other means of systemic delivery may include the following delivery routes: oral, that is, ingested as a tablet, capsule or fluid; inhalation or intranasal; transmucosal or buccal; or transdermal, such as through use of a skin patch. Suspensions or solutions for intramuscular injections may contain together with the active compound, a pharmaceutically acceptable carrier, such as e.g., sterile water, olive oil (or other vegetable or nut derived oil), ethyl oleate, glycols, e.g., propylene glycol, and if so desired, a suitable amount of lidocaine hydrochloride. Adjuvants for triggering the injection effect can be added as well. Solutions for IV injection or infusion may contain as carrier, e.g., sterile water, or preferably, a sterile isotonic saline solution, as well as adjuvants used in the field of injection of active compounds. Such solutions would also be suitable for i.m. and i.c.v. injection.

Induction and Maintenance Regimens

In particular embodiments, the present methods can include the use of a novel regimen comprising an induction regimen followed by a maintenance regimen for administration of one or more TATs. For example, the methods may comprise administering to the subject an induction regimen comprising a therapeutically effective amount of a TAT (e.g., a TNF-I); and administering to the subject a maintenance regimen comprising a therapeutically effective amount of the same or a different TAT. An induction regimen and a maintenance regimen can independently include multiple administrations of a TAT (e.g., 2, 3, 4, 5, 6, 8, 10, or more separate administrations). In some embodiments, a maintenance regimen will comprise more separate administrations of a TAT than an induction regimen. For example, an induction regimen may comprise one administration of a TAT (e.g., a single intradiskal administration), while a maintenance regimen may comprise weekly or monthly intramuscular injections for a period of 1 month, 2 months, 3 months, 6 months to a year, or longer.

The induction regimen can provide for a substantive, rapid, or clinically relevant induction of protection from neuronal insult or remission of pain or other symptoms (e.g., weakness, numbness). Although not being bound by theory, it is believed that the induction regimen can provide for interruption of one or more of the biological and physiological processes which contribute to symptoms such as severe and/or persistent pain, and/or injury, mediated by ICs or IMs. The induction regimen may comprise administering at least one dose (an “induction dose” or “loading dose”) of at least one TAT, e.g., a TNF-I, such that induction of remission of pain or other symptoms, or protection from exacerbation of symptoms occurs.

An induction regimen can involve a more invasive route of administration than a maintenance regimen. A more invasive route of administration can be evaluated according to the invasiveness spectrum defined previously. Thus, an induction regimen can include a mode of administration selected from intrathecal, intradiskal, epidural (including transforaminal and periradicular), or perispinal, while a maintenance regimen can be selected from perispinal (provided the induction regimen is not perispinal), IV, intramuscular, or SC administration. In some cases, an induction regimen will be selected from intradiskal or epidural, while a maintenance regimen will be selected from IV, intramuscular, or SC administration.

An induction regimen can involve a more local or targeted administration than a maintenance regimen. A more local administration can be obtained targeting the administration to the site of injury or in close proximity to the site of injury in the subject. Modes of administration that result in “systemic” administration are understood by those having ordinary skill in the art to be “non-local” and non-targeted. Thus, in some cases, an induction regimen will include administration in proximity to the site of spinal pathology (e.g., site of an HD, SS, adhesion, sensory nerve, or internal disk derangement), while the maintenance regimen will involve non-targeted administration. For example, an induction regimen can involve intradiskal or epidural administration to an HD, site of SS, adhesion, or internal disk derangement, while a maintenance regimen can involve systemic administration, e.g., through IV, intramuscular, or SC administration.

In preferred embodiments, the more local and/or more invasive route of administration of an induction regimen results in a higher concentration of drug in or at the presumed site of therapeutic action or pathology, such as the affected NR.

An induction regimen comprises a lower dose per administration of a TAT than a maintenance regimen. The dose per administration can be evaluated by those having ordinary skill in the art. Typically, the lower dose per administration of an induction regimen is less than about 50% of the maintenance dose per administration, e.g., less than about 50%, 45%, 40%, 35%, 30%, 25%, 20%, 15%, 10%, or 5% of the maintenance dose per administration.

In particular embodiments, an induction regimen may comprise local (e.g., at the site of a HD), invasive administration (e.g., epidural, intradiskal, peridiskal administration) of one or more low doses per administration (low as compared to the maintenance dose per administration) of at least one TAT, e.g., in an amount sufficient to provide clinically meaningful relief of pain or other symptoms. In preferred embodiments, an “induction regimen” comprises one to seven (e.g., 1, 2, 3, 4, 5, 6, or 7) intradiskal or epidural (including periradicular and transforaminal) administrations of at least one TNF-I selected from the group consisting of: Enbrel® (etanercept); Humira® (adalimumab); Humicade® (CDP-570); Cimzia® (certolizumab pegol); Remicade® (infliximab), CNTO-148, Peptech antibody, Wyeth-Trubion SMIP, Wyeth-Ablynx antibody fragement, and PN0621 (mini-antibodies against TNF).

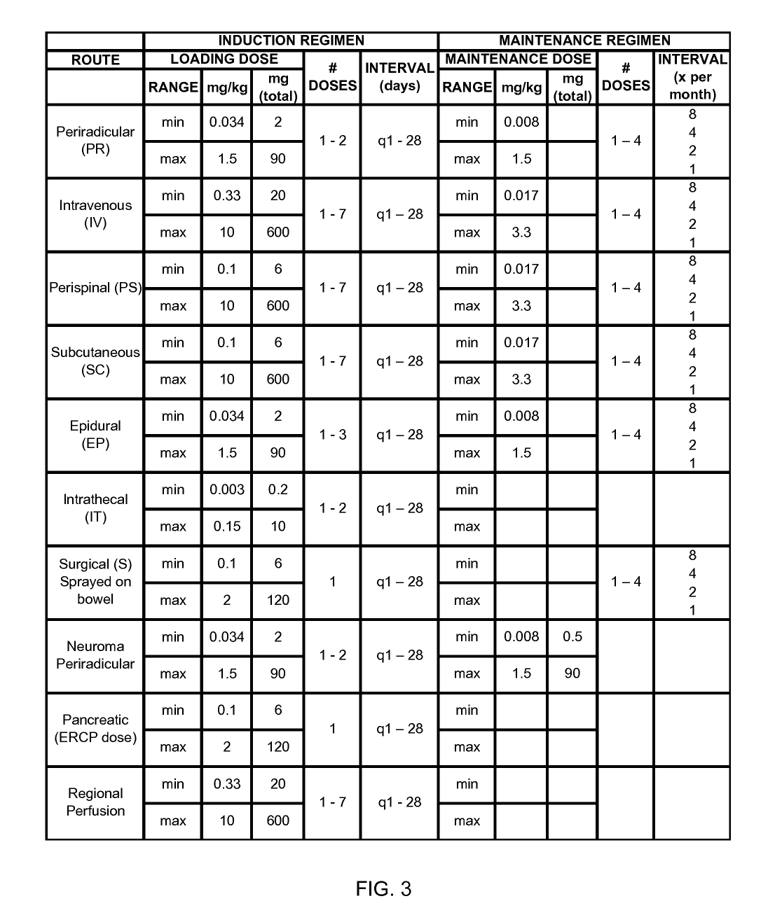

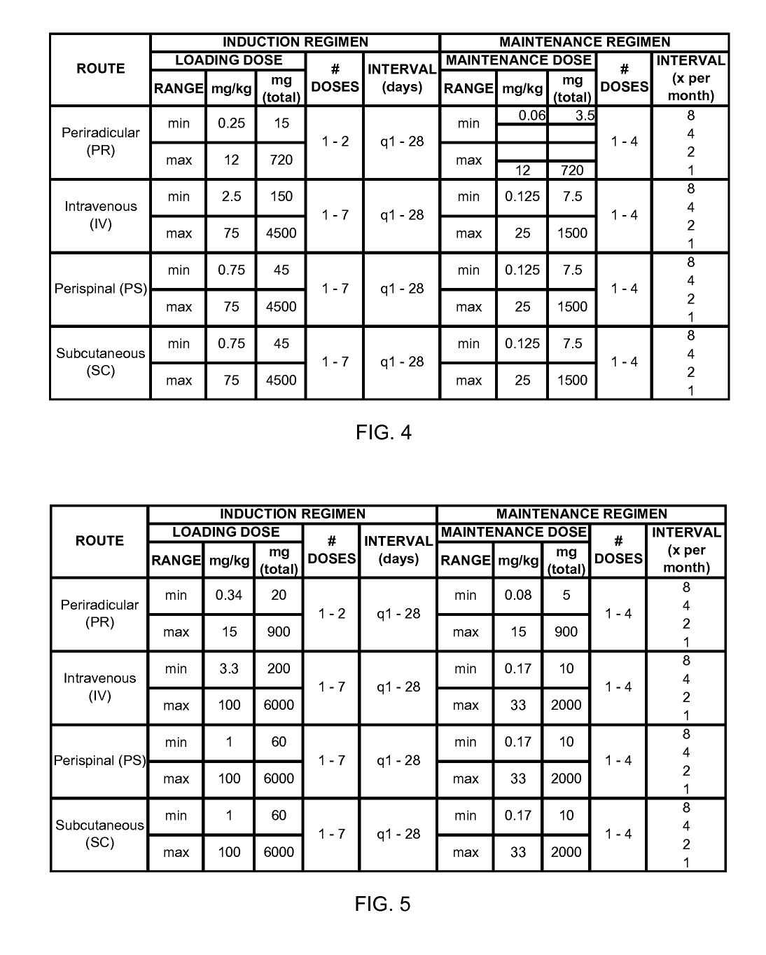

Preferred dosage ranges for an “induction regimen” of a TAT will vary depending upon clinical factors observed by the clinician, the indication, and the particular TAT, and will generally comprise administration of a “loading dose” of at least one TAT, or a dose which will generally achieve clinically meaningful induction of protection from neuronal insult or relief of pain upon administration. In preferred embodiments, the induction regimen will provide protection from injury or relief of pain or other symptoms within several hours of administration. In some embodiments, the induction regimen comprises administration of a “loading dose” of at least one TAT (e.g., TNF-I) via local administration, for example via epidural, intradiskal, intradiskal/peridiskal, intradiskal/epidural or intrathecal administration. Preferred induction regimens for several approved TNF-Is are provided in FIGS. 3-5.

A maintenance regimen can provide for durable protection from neuronal insult or relief from pain or other symptoms similar to the relief afforded by an induction regimen. A maintenance regimen can comprise administration of at least one dose of at least one TAT to maintain such relief for a period of time (e.g., a “maintenance dose”), preferably the period of time being at least one to twenty-four hours, at least twenty-four hours to one week, or at least one week to three months. A maintenance regimen may accompany and/or follow administration of an induction regimen.

A maintenance regimen of a TAT will also vary depending upon clinical factors observed by the clinician, the indication, and the type of inhibitor, and can comprise administration of a “maintenance dose” of at least one TAT (e.g., TNF-I), or a dose which will generally achieve durable induction of relief from pain or protection from exacerbation of symptoms when administered concurrently with and/or subsequent to, administration of an “induction regimen.” A “maintenance regimen” of a TAT may be administered once, or may be administered periodically (e.g., hourly, every 4 hours, every 6 hours, every 12 hours, daily, weekly, monthly, bimonthly) according to a dosage regimen prescribed by the treating physician. The maintenance regimen comprises administration of a maintenance dose of at least one TAT via a less invasive or less local mode of administration than an induction regimen but that is still effective for durable induction of protection from neuronal insult or relief from pain. For example, a maintenance dose of TAT will be administered via less invasive modes of administration, such as IV, intramuscular, or SC administration. In some embodiments, the maintenance regimen comprises administration of at least one maintenance dose via continuous dosage means, such as a pump and catheter. The catheter may be inserted during the course of administering the induction regimen, or may be separately inserted. Preferred maintenance regimens for several approved TNF-Is are provided in FIGS. 3-5.

Routes of administration, timing of administration, and choice of TAT for the “induction regimen” and “maintenance regimen” will vary depending upon the practitioner's choice of regimen, the indication, and the type of inhibitor. The criteria that might lead a skilled practitioner to choose a particular TAT for a particular regimen will often include drug concentration, lipophilicity, solubility, half life, formulation characteristics, pH, pKa, known adverse events profile, tmax, potency, and affinity (e.g., for the target), among other factors. The relative weight and strength of the applicability of each of these criteria would depend, in part, on the indication and on the site of administration. Thus, for example, since a limited volume of agent can be safely injected intradiskally, an agent high in concentration might be chosen to maximize the dosage given. In an epidural route of administration, a lipophilic agent might limit spread of the TAT to distant, non-pathologic locations within the epidural space, while choice of a large protein TAT or a depot formulation might limit migration out of the epidural space. Moreover, in certain embodiments, the induction regimen is administered and completed prior to beginning administration of the maintenance regimen. In others, the maintenance regimen may begin at or near the same time as the induction regimen.

The TAT for use in the maintenance regimen may be the same as or different than the TAT for use in the induction regimen. The formulation of the TATs can be the same or different, e.g., both can be an aqueous formulation, or one could be aqueous while the other is an oil-in-water emulsion, or one could be aqueous while the other could be a depot or controlled-release formulation.

In an embodiment, the induction regimen and/or maintenance regimen may be administered by means of a catheter and pump system, such as a fully implantable pump system or an external pump system. Suitable pump and catheter systems are commercially available, e.g., SynchroMed® pump and InDura® intrathecal catheters (both from Medtronic Sofamor Danek, Memphis, Tenn.). The induction and/or maintenance regimen may also be administered as part of an implantable device that comprises a depot formulation of one or more TATs. In some embodiments, the device comprising a depot formulation may take the form of a biodegradable or resorbable substance, including polymers such as poly lactic acid, (PLA), polyglycolic acid (PGA), a hydrogel, and co-polymers of polylactic acid/polyglycolic acid (PLGA). The device comprising a depot formulation may comprise capsules or microcapsules. In a further embodiment, the maintenance regimen may be administered by transfusion, such as IV transfusion.

### Compositions, Formulations, and Kits

Compositions and Formulations

Also provided herein are compositions and formulations for use in the described methods. Novel compositions or formulations can be based on the need for particular concentration ranges of a TAT or particular formulation characteristics (e.g., lipophilicity, pH, stability) in the administration regimen chosen. For example, provided herein is a pharmaceutical composition comprising a direct TNF-I at a concentration in the range of from about 1 to about 100 mg/cc, e.g., about 5 to about 50 mg/cc. Such a composition can be useful for the more invasive modes of administration contemplated herein, e.g., intradiskal, peridiskal, epidural, and intrathecal administration. The direct TNF-I for use in the formulation can be any of those previously described, and in some cases is selected from adalimumab, infliximab, CDP-870, CDP-570, etanercept, and pegsunercept. Any of the compositions can further include other agents, including the SAIs described previously.

TAT compositions useful in the practice of the invention comprise at least one TAT, and in the case of small molecule inhibitors, its pharmaceutically acceptable salt, solvate, hydrate, stereoisomer, clathrate, or prodrug thereof. The composition, shape, and type of dosage form will typically vary depending on their use. For example, a parenteral dosage form may contain smaller amounts of one or more of the active ingredients than an oral dosage form used for the same purpose. These and other ways in which specific dosage forms encompassed by this invention will vary from one another will be readily apparent to those skilled in the art. See, e.g., [13]. Typical pharmaceutical compositions and dosage forms also comprise one or more excipients. Suitable excipients are well known to those skilled in the art.

The invention further encompasses the use of compounds that reduce the rate by which the TAT or SAI will decompose. Such compounds, which are referred to herein as “stabilizers,” include, but are not limited to, antioxidants such as ascorbic acid, pH buffers, or salt buffers. The amounts and specific types of stabilizers or other ingredients in a dosage form may differ depending on factors such as, but not limited to, the route by which it is to be administered to patients.

Kits

The present disclosure also contemplates kits for use in the methods described herein. In some embodiments, a kit is provided that includes a syringe, catheter, pump, or delivery device, where the syringe, catheter, pump or delivery device is adapted for epidural or intradiskal administration, and a TAT. The TAT can be disposed within the syringe, catheter, pump, or delivery device and/or can be contained in a vial. The kits can further include other optional ingredients, including an SAI, which could in a separate vial from the TAT, in the same vial as the TAT, or disposed within the syringe, catheter, pump, or delivery device. A kit can further include a direct TNF-I disposed within a hydrogel or depot form of administration. In some embodiments, a kit can include a TAT at a concentration in the range of from about 1 to about 100 mg/cc, e.g., in the range of from about 5 to about 50 mg/cc.

In other embodiments, the kit may comprise devices or apparatuses that are used to administer the active ingredients. Examples of such devices include, but are not limited to, syringes, needles, catheters, drip bags, patches and inhalers. In some embodiments, the kit might include, for example, some or all of the necessary syringes, needles, catheters and other disposable equipment useful for intrathecal, intradiskal, peridiskal, or epidural placement and administration, either with or without fluoroscopic guidance. Likewise, the kit might contain the necessary syringes, needles, and tubes for IV administration, or for SC administration of the TAT.

In some embodiments, one or more of the active ingredients in the kit might need to be separated from the other components of the kit and refrigerated until the time that the kit is used.

Kits can include without limitation a first TAT and a second TAT and devices/apparatuses to facilitate delivery by different routes, such as intradiskal/epidural injection or IV infusion. The first and second TAT could be the same or different. Kits of the invention may further comprise pharmaceutically acceptable vehicles that can be used to administer one or more of the active ingredients. For example, if an active ingredient is provided in a solid form that must be reconstituted for IV or SC administration, the kit may comprise a sealed container of a suitable vehicle in which the active ingredient can be dissolved to form a particulate-free sterile solution suitable for parenteral administration. Examples of pharmaceutically acceptable vehicles include, but are not limited to, water for injection USP; aqueous vehicles such as, but not limited to, sodium chloride injection, Ringer's Injection, Dextrose Injection, Dextrose and Sodium Chloride Injection, and Lactated Ringer's Injection; water-miscible vehicles such as, but not limited to, ethyl alcohol, polyethylene glycol and polypropylene glycol; and non-aqueous vehicles such as, but not limited to, corn oil, cottonseed oil, peanut oil, ethyl oleate, isopropyl myristate and benzyl benzoate.

### EXAMPLES

### Example 1

### Subject Eligible for Diskectomy

A subject who is suffering from low back pain and leg pain is seen by his general practitioner (GP), who recommends conservative treatment (e.g., rest, analgesics, physical therapy) for a period of 6 weeks. After 6 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has a HD at L5 and is eligible for a full diskectomy based on the subject meeting the diskectomy eligibility criteria in the NASS CPG for HD [3], including MRI findings of HD at the appropriate level, the persistent pain of the subject for more than 6 weeks, and the failure of conventional conservative treatment. The spine interventionalist, based on the subject's eligibility for the diskectomy, recommends that the subject undergo a course of treatment with a TAT, specifically a direct TNF-I such as infliximab, to delay the need for the surgery or to improve the outcome of the surgery, should it ultimately result. The spine interventionalist administers the direct TNF-I epidurally to the subject. The subject is assessed post-administration using one or more of the following: the Roland disability questionnaire, the Oswetry disability questionnaire, the VAS pain scale, the Likert scale, an MRI evaluation, and a neurological assessment.

### Example 2

### Subject Eligible for Diskectomy

A subject who is suffering from low back pain and leg pain is seen by his GP, who recommends conservative treatment (e.g., rest, analgesics, steroids) for a period of 6 weeks. After 6 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has an HD at L4 and is eligible for a partial diskectomy based on the subject meeting the diskectomy eligibility criteria in the NASS CPG for HD [3], including MRI findings of HD at the appropriate level, the persistent pain of the subject for more than 6 weeks, and the failure of conventional conservative treatment. The spine interventionalist, based on the subject's eligibility for the partial diskectomy, recommends that the subject undergo a course of treatment with a TAT, specifically a direct TNF-I such as infliximab, to delay the need for the surgery or to improve the outcome of the surgery, should it ultimately result. The spine interventionalist administers the direct TNF-I intradiskally and further delivers an epidural and a peridiskal dose to the subject in the vicinity of the HD. The subject is assessed post-administration using one or more of the following: the Roland disability questionnaire, the Oswetry disability questionnaire, the VAS pain scale, the Likert scale, an MRI evaluation, and a neurological assessment.

### Example 3

### Subject Eligible for Diskectomy

A subject who is suffering from neck and arm pain is seen by his GP, who recommends conservative treatment (e.g., rest, analgesics) for a period of 8 weeks. After 8 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has an HD at C2 and is eligible for a full diskectomy based on the subject meeting the diskectomy eligibility criteria in the NASS CPG for HD [3], including MRI findings of HD at the appropriate level, the persistent pain of the subject for more than 8 weeks, and the failure of conventional conservative treatment. The spine interventionalist, based on the subject's eligibility for the diskectomy, recommends that the subject undergo a course of treatment with a TAT, specifically a direct TNF-I such as etanercept, to delay the need for the surgery. The spine interventionalist administers the direct TNF-I intradiskally and peridiskally to the subject. The patient is then placed on a maintenance regimen of weekly SC doses of etanercept. After 20 weeks, the subject returns to the spine interventionalist, complaining of continued symptoms. The spine interventionalist opts to perform the surgery, and interrupts the etanercept injections for 2 weeks prior to surgery. Starting at 1 weeks post surgery, the subject is administered a TAT SC every 1 week for a period of 12 weeks. The subject is assessed post-administration using one or more of the following: the Roland disability questionnaire, the Oswetry disability questionnaire, the VAS pain scale, the Likert scale, an MRI evaluation, and a neurological assessment.

### Example 4

### Subject Eligible for Laminectomy

A subject who is suffering from leg pain, numbness and tingling, and weakness while walking is seen by his GP, who recommends conservative treatment (e.g., rest, analgesics, and an orthotic brace) for a period of 10 weeks. After 10 weeks, the subject returns to the GP, complaining that the symptoms have not resolved. The subject is referred by the GP to a spine interventionalist to determine if the subject should undergo a laminectomy. After evaluating the patient, the spine interventionalist diagnoses the subject as suffering from SS to such an extent that the subject is eligible for a laminectomy, e.g., based on the subject meeting the laminectomy eligibility criteria in the NASS CPG for SS [4], including MRI findings, the persistent pain of the subject for 10 weeks, and the failure of conventional conservative treatment. The spine interventionalist, based on the subject's eligibility for the laminectomy, recommends that the subject undergo an induction/maintenance course of treatment with a TAT, specifically a direct TNF-I such as infliximab, to delay the need for the surgery. For the induction phase, the spine interventionalist administers the direct TNF-I epidurally to the subject, local to the site of the SS. The subject is then administered a maintenance regimen of a direct TNF-I, where the maintenance regimen includes SC injections of a TNF-I every week for a period of 12 weeks, with the dose of each maintenance regimen injection being higher than the initial epidural induction dose. The subject is assessed post-administration using one or more of the following: the Roland disability questionnaire, the Oswetry disability questionnaire, the VAS pain scale, the Likert scale, an MRI evaluation, and a neurological assessment.

### Example 5

### Subject Eligible for Laminectomy

A subject who is suffering from back pain, leg weakness, and increased pain upon standing, is seen by his GP, who recommends conservative treatment (e.g., rest, analgesics) for a period of 8 weeks. After 8 weeks, the subject returns to the GP, complaining that the symptoms have not resolved. The subject is referred by the GP to a spine interventionalist to determine if the subject should undergo a laminectomy. After evaluating the patient, the spine interventionalist determines that the patient is eligible for a laminectomy based on the subject meeting the laminectomy eligibility criteria in the NASS CPG for SS [4], including MRI findings of SS at the appropriate level, the persistent pain of the subject for more than 8 weeks, and the failure of conventional conservative treatment. The spine interventionalist, based on the subject's eligibility for the laminectomy, recommends that the subject undergo a course of treatment with a TAT, specifically a direct TNF-I such as etanercept, to improve the outcome of the surgery. At a time period of 2 weeks before the laminectomy, the spine interventionalist administers the direct TNF-I intradiskally and peridiskally to the subject, in the region of the stenosis. After 2 weeks, the spine interventionalist then performs the surgery, and starting at 1 week post surgery, the subject is administered a TAT SC every 1 week for a period of 12 weeks. The post-surgery SC doses are all at a higher dose per administration than the pre-surgery dose. The subject is assessed post-administration using one or more of the following: the Roland disability questionnaire, the Oswetry disability questionnaire, the VAS pain scale, the Likert scale, an MRI evaluation, and a neurological assessment.

### Example 6

### Subject Eligible for Epidural Adhesiolysis

A subject experienced a sharp pain radiating into his right leg after lifting a heavy object. He notes a dull aching quality to the pain, and takes some non prescription non-steroidal anti inflammatory agents before going to bed. He awakes in the morning to find the pain persistent and radiating particularly into the medial side of the big toe on his right side. Because the pain is severe he makes an appointment with his GP, who notes the pain to be consistent with NR irritation and refers him to a neurosurgeon. The neurosurgeon confirms signs of a right-sided L4 NR pathology, with positive femoral stretch test and some diminished sensation and patellar reflex asymmetry on this side, and obtains an MRI, which reveals a right-sided large disk bulge compressing the R L4 NR. Surgery is scheduled, and although initially successful with a relief of pain, the subject begins to note some increased pain about 4 weeks following the surgery, in the same distribution, lower in intensity, but also into his leg and thigh. Despite treatment with NSAIDs, opioids, gabapentin and desipramine, the pain persists and the patient is referred to a pain specialist. This doctor diagnoses post-laminectomy pain and schedules R sides L4 transforaminal injections of local anesthestic and steroid, which help temporarily. The symptoms return, despite maximal medication course. The subject is thus determined to be eligible for epidural adhesiolysis and based on the subject's eligibility for the procedure, the pain physician recommends that the subject undergo a course of treatment with a TAT, specifically a direct TNF-I such as etanercept, to improve the outcome of the adhesiolysis. At a time period of 2 weeks before a scheduled epidural adhesiolysis, the pain physician administers the direct TNF-I via a transforaminal manner in the region of the R L4 NR. Two weeks after the adhesiolysis, the pain physician sees the patient and performs a second transforaminal epidural injection of the TNF-I in a similar manner to the first injection. The subject is assessed post-administration using one or more of the following: the Roland disability questionnaire, the Oswetry disability questionnaire, the VAS pain scale, the Likert scale, an MRI evaluation, and a neurological assessment.

### Example 7

### Subject Eligible for Radiofrequency Medial Branch Neurotomy of Lumbar Zygapophyseal Joints

A subject visits his GP, complaining of right sided back pain that is noticeably worse when sitting for long periods. The pain is felt in his side in his mid back and radiates into the R buttock on this side and the back of the upper part of his leg but does not go below the knee. He can reproduce the pain by straight bending to the R side, but it is relieved with bending to the L side. On examination there is point tenderness in the R paramedian area overlying the L2-3 facet joint. CT scan confirms facet joint degeneration at this level. A diagnostic and therapeutic injection of LAs and steroids provides complete relief for one month, but the painful syndrome returns after this time, without provocation. A second injection of a different LA and steroid similarly produces relief of about 4 weeks. Because of the temporary nature of relief and the specificity of the diagnosis, the subject is eligible to undergo radiofrequency lesioning of the sensory nerves to the facet joint. Based on the subject's eligibility for the procedure, the pain physician recommends that the subject undergo a course of treatment with a TAT, specifically a direct TNF-I such as etanercept, to improve the outcome of the radiofrequency neurotomy. At a time period of 2 weeks before a scheduled RFN, the pain physician administers the direct TNF-I into the right L2-3 facet joint under fluoroscopic guidance. Two weeks after the procedure, the pain physician sees the patient and performs a second intra-articular facet joint injection of the TNF-I in a similar manner to the first injection. The subject is assessed post-administration using one or more of the following: the Roland disability questionnaire, the Oswetry disability questionnaire, the VAS pain scale, the Likert scale, an MRI evaluation, and a neurological assessment.

### Example 8

### Subject Eligible for Diskectomy

A subject who is suffering from low back pain and leg pain is seen by his GP, who recommends conservative treatment (e.g., rest, analgesics, steroids) for a period of 8 weeks. After 8 weeks, the subject returns to the GP, complaining that the pain has not resolved. The subject is referred by the GP to a spine interventionalist to determine if the subject should undergo a partial or full diskectomy. After evaluating the patient, the spine interventionalist determines that the patient has an HD at L4 and is eligible for a diskectomy based on the subject meeting the diskectomy eligibility criteria in the NASS CPG for HD [3], including MRI findings of HD at the appropriate level, the persistent pain of the subject for more than 8 weeks, and the failure of conventional conservative treatment. The spine interventionalist, based on the subject's eligibility for a diskectomy, recommends that the subject undergo a combination therapy combining the administration of a TAT, specifically a direct TNF-I such as etanercept, and the administration of medical ozone therapy. The spine interventionalist administers the direct TNF-I transforaminally at the L4 NR on the affected side and further administers a dose of the TNF-I in a midline translaminar approach at the L4-L5 interspace of the subject in the vicinity of the HD. While the needle is inserted, a portable ozone generator is used to administer ozone to the patient's disk. The subject is assessed post-administration using one or more of the following: the Roland disability questionnaire, the Oswetry disability questionnaire, the VAS pain scale, the Likert scale, an MRI evaluation, and a neurological assessment.

A number of embodiments of the invention have been described. Nevertheless, it will be understood that various modifications may be made without departing from the spirit and scope of the invention. Accordingly, other embodiments are within the scope of the following claims.

### REFERENCES

1 Shin K C et al. (2005). A prospective controlled trial of TNF-alpha inhibitor for symptomatic patients with cervical disk herniation. Spine J. 5: 45S.
2 Korhonen T et al. (2005). The treatment of disc-herniation-induced sciatica with infliximab: results of a randomized, controlled, 3-month follow-up study. Spine 30:2724-28.
3 North American Spine Society (2000). Phase III Clinical Guidelines for Multidisciplinary Spine Care Specialists. Herniated Disc Version 1.0. NASS: La Grange, Ill.
4 North American Spine Society (2002). Phase III Clinical Guidelines for Multidisciplinary Spine Care Specialists. Spinal Stenosis Version 1.0. NASS: La Grange, Ill.
5 Trescot A M et al. (2007). Systematic Review of Effectiveness and Complications of Adhesiolysis in the Management of Chronic Spinal Pain: An Update. Pain Phys. 10:129-146.
6 Boswell M V et al. (2007). Interventional techniques: evidence-based practice guidelines in the management of chronic spinal pain. Pain Phys. 10:7-111.
7 Armour K L et al. (1999). Recombinant human IgG molecules lacking Fcgamma receptor I binding and monocyte triggering activities. Eur. J. Immunol. 29:2613-2624.
8 De A U and Pal D (1975). Possible antineoplastic agents I. J. Pharm. Sci. 64(2):262-266.
9 List A et al. (2005). Efficacy of Lenalidomide in Myelodysplastic Syndromes. N. Eng. J. Med. 352:549-57.
10 Muto M (2007). Intradiscal and Intraforaminal Oxygen-ozone Therapy in the Treatment of Herniated Disks. Abstract presented at the American Society of Spine Radiology Annual Symposium, February 22-25 in Marco Island, Fla.
11 PDF Staff (2003). Physicians' Desk Reference, 57^{th} ed. 594-597. Montvale, N.J.: Thomson Healthcare.
12 Lamer T J (1994). Mayo Clinic Proceedings. 69:473-480.
13 Gennard, A R, editor. (1990). Remington's Pharmaceutical Sciences, 18th edition. Easton, Pa.: Mack Publishing Company.

1.-11. (canceled)

12. A method for preventing or postponing a spinal surgery procedure in a subject wherein the subject meets at least one predetermined standard of eligibility (SOE) for a spinal surgery procedure, the method comprising:
a) identifying the subject as a subject eligible for the spinal surgery procedure;
b) administering to the subject a therapeutically effective amount of at least one direct TNF-I; and
c) determining whether the subject's eligibility for the spinal surgery procedure has been prevented or postponed.

13. (canceled)

14. The method of claim 12, wherein the subject is:
a) diagnosed with herniated disk (HD) and is eligible for diskectomy; or
b) diagnosed with spinal stenosis (SS) and is eligible for laminectomy.

15.-18. (canceled)

19. The method of claim 12, wherein the direct TNF-I is administered locally to an HD or site of SS.

20. (canceled)

21. The method of claim 12, wherein the route of administration is selected from the group consisting of intra-operative, intrathecal, intradiskal, peridiskal, epidural (including periradicular and transforaminal), any combination of intradiskal, epidural, and peridural, perispinal, IV, intramuscular, subcutaneous (SC), oral, intranasal, inhalation, and transdermal.

22. The method of claim 12, wherein the administration in b) treats the subject so that the subject does not undergo a spinal surgery procedure in at least the first three months after the initial administration of the TNF-I.

23.-30. (canceled)

31. The method of claim 12, wherein the direct TNF-I is selected from the group consisting of an antibody or antibody fragment, a fusion protein, a peptide, a small modular immuno pharmaceutical (SMIP), a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

32. The method of claim 31, wherein the oligonucleotide is an siRNA.

33. The method of claim 31, wherein the direct TNF-I is selected from the group consisting of: Humira® (adalimumab/D2E7); Remicade® (infliximab); Cimzia® (CDP-870); Humicade®(CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

34. (canceled)

35. The method of claim 12, wherein the administration comprises: (a) an induction regimen comprising a direct TNF-I; and (b) a maintenance regimen comprising a direct TNF-I.

36. (canceled)

37. The method of claim 35, wherein the induction regimen is administered intrathecally, intradiskally, peridiskally, or epidurally, or combinations thereof.

38. The method of claim 35, wherein the maintenance regimen comprises systemic or parenteral administration.

39.-44. (canceled)

45. The method of claim 35, wherein the induction regimen is administered locally to an HD or site of SS, and wherein the maintenance regimen is administered systemically or parenterally.

46.-50. (canceled)

51. A method for improving the outcome of a spinal surgery procedure in a subject, wherein the subject meets at least one predetermined SOE for a spinal surgery procedure, the method comprising:
a) identifying the subject as a subject eligible for the spinal surgery procedure;
b) administering to the subject a therapeutically effective amount of at least one direct TNF-I; and
c) performing the spinal surgery procedure.

52. (canceled)

53. The method of claim 51, wherein the subject is:
a) diagnosed with HD and is eligible for diskectomy; or
b) diagnosed with SS and is eligible for laminectomy.

54. (canceled)

55. The method of claim 51, wherein said administration of a direct TNF-I is in a time period that can be one or more of prior to, during, or after the time period of the spinal surgery procedure.

56.-60. (canceled)

61. The method of claim 51, wherein the direct TNF-I is selected from the group consisting of an antibody or antibody fragment, a fusion protein, a peptide, a SMIP, a small molecule, an oligonucleotide, an oligosaccharide, a soluble cytokine receptor or fragment thereof, a soluble TNF receptor Type I or a functional fragment thereof, a polypeptide that binds to TNF, and a dominant negative TNF molecule.

62. The method of claim 61, wherein the oligonucleotide is an siRNA.

63. The method of claim 61, wherein the direct TNF-I is selected from the group consisting of: Humira® (adalimumab/D2E7); Remicade® (infliximab); Cimzia® (CDP-870); Humicade® (CDP-570); golimumab (CNTO 148); CytoFab (Protherics); AME-527; anti-TNF-Receptor 1 mAb or dAb; ABX-10131; polyclonal anti-TNF antibodies; anti-TNF polyclonal anti-serum; anti-TNF or anti-TNF-R SMIPs (Trubion); Enbrel® (etanercept); pegsunercept/PEGs TNF-R1, onercept; recombinant TNF binding protein (r-TBP-1); trimerized TNF antagonist; SSR-150106 (Sanofi-Synthelabo); ABX-0402 (Ablynx); nanobody therapeutics (Ablynx); trimerized TNF antagonist (Borean); humanized anti-TNF mAb (Biovation); Dom-0200 (Domantis); Genz-29155 (Genzyme); agarooligosaccharide (Takara Shuzo); HTDN-TNF (Xencor); and therapeutic human polyclonal anti-TNF and anti-TNF-R antibodies (THP).

64. (canceled)

65. The method of claim 51, wherein the administration comprises: (a) an induction regimen comprising a direct TNF-I and (b) a maintenance regimen comprising a direct TNF-I.

66. (canceled)

67. A kit comprising a syringe, catheter, pump, or delivery device, wherein the syringe, catheter, pump or delivery device are adapted for epidural, intradiskal, or peridiskal administration, or any combination thereof, and a direct TNF-I.

68.-76. (canceled)
